(19) United States
(12) Patent Application Publication
(10) Pub. No.: US 2023/0295667 A1
(43) Pub. Date: Sep. 21, 2023

(54) USE OF ANTI-CRISPR AGENTS TO CONTROL EDITING IN HUMAN EMBRYOS

(71) Applicant: The Trustees of Columbia University in the City of New York, New York, NY (US)

(72) Inventor: Dietrich Egli, New York, NY (US)

(21) Appl. No.: 18/121,759

(22) Filed: Mar. 15, 2023

Related U.S. Application Data
(60) Provisional application No. 63/319,963, filed on Mar. 15, 2022.

(51) Int. Cl. C12N 15/90; C12N 15/10

(52) U.S. Cl. CPC C12N 15/907; C12N 15/102; C12N 2310/20

(57) ABSTRACT

The present disclosure relates to using anti-CRISPR agents and methods to control, reduce and/or inhibit gene editing, thus reducing, eliminating and/or preventing mosaicism and off-target effects of gene editing in embryos. The gene editing can be performed using CRISPR technology and the CRISPR technology can be a CRISPR/Cas9 system.

### CROSS REFERENCE TO RELATED APPLICATION

The present application claims priority to U.S. Provisional Applications No. 63/319,963 filed on Mar. 15, 2022, which is incorporated by reference herein in its entirety.

### FIELD

The present disclosure relates to using anti-CRISPR agents and methods to control, reduce and/or inhibit gene editing, thus reducing, eliminating and/or preventing mosaicism and off-target effects of gene editing in embryos.

### BACKGROUND

CRISPR (clustered regularly interspaced short palindromic repeats) is a revolutionary gene-editing technology that uses a bacterial-derived DNA endonuclease to induce double-strand breaks (DSB) at precise locations in the genomes of model organisms (Rodriguez-Rodriguez et al. 2019). The precise genetic modifications make it, a promising tool to potentially correct disease-causing mutations. Prior studies have attempted to use CRISPR to target several diseases in somatic cells, such as cystic fibrosis, severe combined immunodeficiency (SCID), chronic myeloid leukemia, and sickle cell disease (Rodriguez-Rodriguez et al. 201.9). Moreover, the application of CRISPR in human embryos may prevent disease-causing mutations from propagating in the human germline (Turocy et al. 2021).

The timing of CRISPR-Cas9 activity plays a significant role in the uniformity of gene editing. Cas9 cleavage and repair occurring before the first S-phase would lead to uniform editing, as there is a single copy of each chromosome (Mehravar et al. 2019). Cas9 activity after the first S-phase increases the likelihood of mosaicism, as there are four potential alleles to target in autosomes.

Given the potential consequences of unintended gene editing, there is a need for regulatory mechanisms to control its activity. Inhibitors of CRISPR, termed anti-CRISPR proteins, were first discovered in viruses in 2013 (Davidson et al. 2020). Since then, several anti-CRISPR families have been used to disable or prevent Cas9-mediated gene editing in different model organisms. Shin et al. 2017 showed that the anti-CRISPR protein, AcrIIA4 was able to bind and inhibit CRISPR-Cas9 activity in human cells. Moreover, the delayed introduction of AcrIIA4 into human cells allowed on-target gene editing while significantly reducing off-target edits, effectively serving as an “off-switch” to Cas9 clea-vage. A later study by Lee et al. 2019 demonstrated that anti-CRISPR proteins also work in viva in somatic cells of mice.

Cas9-mediated gene editing is a promising tool for the treatment of heritable genetic diseases of the human germline. However, limitations remain before this technology can be used clinically, most notably mosaicism, chromosomal loss at Cas9 cut sites, and off-target editing. Different strategies have been developed to try and overcome some of these obstacles in somatic cells, including the use of modified Cas9 variants to reduce the timing of Cas9 activity (Tu et al. 2017), the use of DNA replication inhibitors to prolong the time before the S-phase (Nacarro-Serna et al. 2022), and the use of anti-CRISPR proteins to regulate Cas9 activity in human cells (Shin et al. 2017). However, no studies to date have attempted to control CRISPR activity in human embryos. Prior studies have demonstrated that off-target gene editing is delayed relative to on-target gene editing, pointing to a narrow window in which Cas9 activity is more likely to be specific (Zuccaro et al. 2020; Shin et al. 2017). Similarly, mosaicism is less likely to occur if Cas9 is active before the first DNA replication cycle and not after (Mehravar et al. 2019). Chromosome loss remains a potential issue as its origin appears to be related to the inability of the target genome to repair the DSB and not a temporal factor.

To improve the success of gene editing in the human embryo, it is important to ascertain the kinetics of CRISPR-mediated cleavage. In an ideal system, CRISPR should induce DSBs rapidly before the first S phase to edit on-target sites and be turned off shortly after to minimize the risk of mosaicism and unintended off-target editing.

Disclosed herein agents and methods which prevent mosaicism and off-target effects during gene editing of embryos, as well as increase efficiency of gene editing.

### SUMMARY

Disclosed and described herein are agents and methods to control, reduce and/or inhibit gene editing. In some embodiments, the gene editing is performed using CRISPR technology. In some embodiments, the CRISPR technology is a CRISPR/Cas9 system.

In some embodiments, the gene editing is performed in an embryo. In some embodiments, the embryo is human. In some embodiments, the embryo is a non-human animal.

In some embodiments, the agent is selected from the group consisting of small molecules, proteins and polypeptides, and nucleic acids.

In some embodiments, the agent is an anti-CRISPR agent.

In some embodiments, the anti-CRISPR agent is a small molecule.

In some embodiments, the anti-CRISPR agent is an anti-CRISPR protein. In some embodiments, the anti-CRISPR protein is a biological equivalent of an anti-CRISPR protein. In some embodiments, the anti-CRISPR protein is a variant of an anti-CRISPR protein.

In some embodiments, the anti-CRISPR protein is selected from the group consisting of the proteins listed in Table 1. In some embodiments, the anti-CRISPR protein is a biological equivalent of a protein listed in Table 1. In some embodiment, the anti-CRISPR protein is a variant of a protein listed in Table 1.

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the agents are administered to the embryo. In some embodiments, the embryo is human. In some embodiments, the embryo is a non-human animal.

In some embodiments, the agents control, reduce and/or inhibit the activity of the gene-editing.

In some embodiments, the agents reduce, eliminate and/or prevent mosaicism.

In some embodiments, the agents reduce, eliminate and/or prevent off-target gene editing.

A further embodiment of the present disclosure is a method of controlling gene editing in an embryo comprising introducing an anti-CRISPR agent into the embryo after the gene editing.

A further embodiment of the present disclosure is a method of reducing and/or inhibiting gene editing in an embryo comprising introducing an anti-CRISPR agent into the embryo after the gene editing.

A further embodiment of the present disclosure is a method of reducing, eliminating and/or preventing mosaicism in an embryo comprising introducing an anti-CRISPR agent into the embryo after the gene editing.

Yet a further embodiment of the present disclosure is a method of reducing, eliminating and/or preventing off-target gene editing in an embryo comprising introducing an anti-CRISPR agent into the embryo after the gene editing.

A further embodiment of the present disclosure is a method of increasing gene editing efficiency in an embryo comprising introducing an anti-CRISPR agent into the embryo after the gene editing.

In some embodiments, the embryo is human. In some embodiments, the embryo is a non-human animal.

In some embodiments, the gene editing is performed using CRISPR technology.

In some embodiments, the CRISPR technology is a CRISPR/Cas9 system.

In some embodiments, the gene editing in the embryo takes place in the first cell cycle.

In some embodiments, the anti-CRISPR agent is selected from the group consisting of small molecules, proteins and polypeptides, and nucleic acids.

In some embodiments, the anti-CRISPR agent is a small molecule.

In some embodiments, the anti-CRISPR agent is an anti-CRISPR protein. In some embodiments, the anti-CRISPR protein is a biological equivalent of an anti-CRISPR protein. In some embodiments, the anti-CRISPR protein is a variant of an anti-CRISPR protein.

In some embodiments, the anti-CRISPR protein is selected from the group consisting of the proteins listed in Table 1. In some embodiments, the anti-CRISPR protein is a biological equivalent of a protein listed in Table 1. In some embodiment, the anti-CRISPR protein is a variant of a protein listed in Table 1.

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the introduction of the anti-CRISPR agent into the embryo is after a time sufficient to allow for the gene editing of a targeted locus or allele. In some embodiments, the gene editing takes place in the first cell cycle. In some embodiments, the introduction of the anti-CRISPR agent is during the first cell cycle. In some embodiments, the introduction of the anti-CRISPR agent is in advance of, or before, the replication of a targeted locus or allele.

In some embodiments, the introduction of the anti-CRISPR agent is about 24 hours after the introduction of the gene editing system or agent into the embryo. In some embodiments, the introduction of the anti-CRISPR agent is less than 24 hours after the introduction of the gene editing system or agent into the embryo. In some embodiments, the introduction of the anti-CRISPR agent is about 12 to about 20 hours after the introduction of the gene editing system or agent into the embryo. In some embodiments, the introduction of the anti-CRISPR agent is about 14 to about 18 hours after the introduction of the gene editing system or agent into the embryo. In some embodiments, the introduction of the anti-CRISPR agent is about 16 to about 18 hours after the introduction of the gene editing system or agent into the embryo. In some embodiments, the introduction of the anti-CRISPR agent is about 16 hours after the introduction of the gene editing system or agent into the embryo.

Alternatively, the introduction of the anti-CRISPR agent is less than one hour after gene editing has taken place in the embryo, or about one hour after gene editing has taken place in the embryo, or about one hour to about four hours after gene editing has taken place in the embryo, or about four hours to about eight hours after gene editing has taken place in the embryo, or about eight hours after gene editing has taken place in the embryo.

The current disclosure also provides for a method of performing gene editing in an embryo comprising introducing into the embryo at least one guide RNA or DNA encoding at least one guide RNA, wherein the guide RNA targets a locus or allele, and an RNA-guided endonuclease, or a nucleic acid encoding the RNA-guided endonuclease, and further comprising introducing an anti-CRISPR agent after the introduction the at least one guide RNA, or DNA encoding at least one guide RNA and the RNA-guided endonuclease, or the nucleic acid encoding the RNA-guided endonuclease, after a time sufficient to allow for gene editing in the targeted locus or allele, wherein the anti-CRISPR agent inhibits or reduces gene editing effects of the at least one guide RNA, or DNA encoding at least one guide RNA and/or the RNA-guided endonuclease, or the nucleic acid encoding the RNA-guided endonuclease.

In some embodiments, the embryo is human. In some embodiments, the embryo is a non-human animal.

In some embodiments, the introduction of the anti-CRISPR agent reduces, eliminates and/or prevents mosaicism and/or off-target gene editing and/or increases the efficiency of the gene editing in the embryo.

In some embodiments, the gene editing is performed using CRISPR technology.

In some embodiments, the CRISPR technology is a CRISPR/Cas9 system.

In some embodiments, the anti-CRISPR agent is selected from the group consisting of small molecules, proteins and polypeptides, and nucleic acids.

In some embodiments, the anti-CRISPR agent is a small molecule.

In some embodiments, the anti-CRISPR agent is an anti-CRISPR protein. In some embodiments, the anti-CRISPR protein is a biological equivalent of an anti-CRISPR protein. In some embodiments, the anti-CRISPR protein is a variant of an anti-CRISPR protein.

In some embodiments, the anti-CRISPR protein is selected from the group consisting of the proteins listed in Table 1. In some embodiments, the anti-CRISPR protein is a biological equivalent of a protein listed in Table 1. In some embodiment, the anti-CRISPR protein is a variant of a protein listed in Table 1.

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the introduction of the anti-CRISPR agent is after a time sufficient to allow for the gene editing of a targeted locus or allele. In some embodiments, the gene editing takes place in the first cell cycle. In some embodiments, the introduction of the anti-CRISPR agent is during the first cell cycle. In some embodiments, the introduction of the anti-CRISPR agent is in advance of, or before, the replication of a targeted locus or allele.

In some embodiments, the introduction of the anti-CRISPR agent is about 24 hours after the introduction of the at least one guide RNA, or DNA encoding at least one guide RNA and the RNA-guided endonuclease, or the nucleic acid encoding the RNA-guided endonuclease. In some embodiments, the introduction of the anti-CRISPR agent is less than 24 hours after the introduction of the at least one guide RNA, or DNA encoding at least one guide RNA and the RNA-guided endonuclease, or the nucleic acid encoding the RNA-guided endonuclease. In some embodiments, the introduction of the anti-CRISPR agent is about 12 to about 20 hours after the introduction of the at least one guide RNA, or DNA encoding at least one guide RNA and the RNA-guided endonuclease, or the nucleic acid encoding the RNA-guided endonuclease. In some embodiments, the introduction of the anti-CRISPR agent is about 14 to about 18 hours after the introduction of the at least one guide RNA, or DNA encoding at least one guide RNA and the RNA-guided endonuclease, or the nucleic acid encoding the RNA-guided endonuclease. In some embodiments, the introduction of the anti-CRISPR agent is about 16 to about 18 hours after the introduction of the at least one guide RNA, or DNA encoding at least one guide RNA and the RNA-guided endonuclease, or the nucleic acid encoding the RNA-guided endonuclease. In some embodiments, the introduction of the anti-CRISPR agent is about 16 hours after the introduction of the at least one guide RNA, or DNA encoding at least one guide RNA and the RNA-guided endonuclease, or the nucleic acid encoding the RNA-guided endonuclease.

Alternatively, the introduction of the anti-CRISPR agent is less than one hour after gene editing has taken place in the embryo, or about one hour after gene editing has taken place in the embryo, or about one hour to about four hours after gene editing has taken place in the embryo, or about four hours to about eight hours after gene editing has taken place in the embryo, or about eight hours after gene editing has taken place in the embryo.

In other embodiments, the molecules are introduced into the embryo by microinjection.

In some embodiments, the RNA-guided endonuclease introduces a double-stranded break in a targeted site of the target allele. In some embodiments, the gene editing is performed in the embryo to correct a mutation in the targeted locus or allele. In some embodiments, the mutation is a frame shift mutation and the endonuclease introduces a double-stranded break in a targeted site on the mutated allele resulting in the correction or modification of the frame shift mutation.

In some embodiments, the mutated allele has a known phenotype. Since the phenotype is known, a father or mother or the subject who has the mutated allele would have the corresponding phenotype. Thus, the identification of the allele is within the skill of the art.

Phenotypes, i.e., disorders or diseases, that can be corrected using the methods and systems of the current disclosure include but are not limited to mutations in the EYS locus, Duchenne's Muscular Dystrophy (DMD) locus, and cardiac myosin-binding protein C3 (MYBPC3) locus.

In some embodiments, the double stranded break in the allele is repaired by a MMEJ repair process. In some embodiments, the double stranded break in the allele is repaired by a NHEJ repair process.

In some embodiments, the gRNA is designed to target the wild-type allele.

In some embodiments, the gRNA is designed to target the mutated but not wild-type allele. In some embodiments, the mutated allele is the paternal allele. In some embodiments, the mutated allele is the maternal allele. In some embodiments, the mutated and wild-type allele differ at the PAM sequence motif. In some embodiments, the gRNA is designed such that placement results in cleavage between two identical regions of polynucleotides in the mutated allele, which defines the sites of micro-homology. In some embodiments, these regions are about 2 bps to about 3 bps. In some embodiments, these regions are about 3 bps to about 5 bps. In some embodiments, these regions are about 5 bps to about 8 bps.

In some embodiment, the RNA-guided endonuclease is a Cas nuclease. In some embodiments, the Cas nuclease is Cas9.

In some embodiments, the Cas9 endonuclease originates from Streptococcus pyogenes ((Spy) CRISPR-Cas9 system), and the anti-CRISPR protein is selected from the group consisting of AcrIIA2 and AcrIIA4.

In some embodiments, the RNA-guided endonuclease and gRNA are introduced into the cell or embryo in the form of a ribonucleoprotein complex comprising the endonuclease complexed to least one gRNA. Preparation of such RNP complexes is known in the art or can be obtained commercially.

In some embodiments, the RNP is introduced into an oocyte at the same time as a sperm cell. This can be accomplished using intracytoplasmic sperm injection (ICSI).

In some embodiments, genotyping an oocyte and sperm donor is performed to determine the location of the mutated allele and the specific frame shift mutation on the mutated allele, prior to the introduction of the at least one guide RNA or DNA encoding at least one guide RNA, and the RNA-guided endonuclease, or the nucleic acid encoding the RNA-guided endonuclease the embryo.

The current disclosure also includes kits comprising the agents and composition disclosed herein and for performing the methods disclosed herein.

### BRIEF DESCRIPTION OF THE DRAWINGS

For the purpose of illustrating the invention, certain embodiments of the invention are depicted in drawings. However, the invention is not limited to the precise arrangements and instrumentalities of the embodiments depicted in the drawings.

FIGS. 1A-1B—AcrIIA4 inhibits Cas9 cleavage in human embryos

FIG. 1A is a schematic of the experiment with AcrIIA4 injected at fertilization together with Cas9. FIG. 1B shows the efficiency of indels detected by on target PCR and Sanger sequencing in embryos injected with Cas9, with and without AcrIIA4 at six loci. ***p<0.0001.

FIGS. 2A-2D—The timing of Cas9 cleavage in human embryos

FIG. 2A is a schematic of experiment with Cas9 injected at ICSI and zygotes harvested at the 1 cell stage to assess gene editing. FIG. 2B shows the efficiency of Cas9-mediated indels by on target PCR and Sanger sequencing at 12 or 16 hours after fertilization at seven distinct loci using IDTDNA Cas9. FIG. 2C is a comparison of Cas9 mediated indels at 12 hours, 16 hours, and 72 hours post-fertilization using IDTDNA Cas9. FIG. 2D shows the efficiency of Cas9-mediated indels at 16 hours and 72 hours using Synthego/NEB Cas9 RNP. ***p<0.0001

FIGS. 3A-3G—Delayed injection of AcrIIA4 in zygotes exposed to Cas9 reduces both on and off target editing

FIG. 3A is a schematic of the experiment with Cas9 injected at the time of ICSI and AcrIIA4 injected 16 hours post-fertilization. FIG. 3B shows the efficiency of Cas9 mediated indels at on and off target loci with and without delayed AcrIIA4 using IDTDNA Cas9. FIG. 3C shows the percentage of on target and off target chromosomes with a segmental change at the Cas9 cut site with and without AcrIIA4 using IDTDNA Cas9. FIG. 3D shows the fold inhibition of total edits (indels+chromosomal segmental changes) at on and off target sites using the delayed injection of AcrIIA4 and IDTDNA Cas9. FIG. 3E shows the efficiency of Cas9 mediated indels at on and off target loci with and without delayed AcrIIA4 using Synthego/NEB Cas9.

FIG. 3F shows the percentage of on target and off target chromosomes with a segmental change at the Cas9 cut site with and without AcrIIA4 Synthego/NEB Cas9. FIG. 3G shows the fold inhibition of total edits (indels+chromosomal segmental changes) at on and off target sites using the delayed injection of AcrIIA4 and Synthego/NEB Cas9. ***p<0.001

FIGS. 4A-4B—Cas9 editing after the first cell cycle

FIG. 4A shows the percentage of alleles demonstrating Cas9 activity after the first cell cycle in all edited embryos exposed to Cas9 or Cas9+AcrIIA4 coinjection (defined as the presence of three or more unique genotypes per embryo per loci). FIG. 4B shows the percentage of alleles with Cas9 activity after the first cell cycle in all edited embryos exposed to Cas9 alone or Cas9+delayed AcrIIA4. **p<0.01.

FIGS. 5A-5B—AcrIIA4 reduces mosaicism rates

FIG. 5A shows the percentage of mosaic alleles in all edited embryos exposed to Cas9 or Cas9+AcrIIA4 coinjection (mosaicism defined as the presence of two or more unique genotypes per embryo per loci). FIG. 5B shows the percentage of mosaic alleles in all edited embryos exposed to Cas9 only or Cas9+delayed AcrIIA4.

### DETAILED DESCRIPTION

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

The term “cell” as used herein may refer to either a prokaryotic or eukaryotic cell, optionally obtained from a subject or a commercially available source.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” or homology (equivalence or equivalents) to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid”, “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “variant” of a protein, peptide or polypeptide means a protein, peptide or polypeptide that has an amino acid sequence which differs by one or more amino acids from the polypeptide sequence.

A “variant” of a polynucleotide sequence is defined as a polynucleotide sequence which differs from the reference polynucleotide sequence by having deletions, insertions and/or substitutions.

The terms “edit”, “editing” and the like refer to a method of altering a nucleic acid sequence of a polynucleotide by selective deletion of a specific genomic target or the specific inclusion of a specific new sequence through the use of an exogenously supplied DNA template.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which target polynucleotide sequence is changed through introduction of deletions, insertions, or base substitutions to the polynucleotide sequence. Gene regulation refers to increasing or decreasing the production of specific gene products such as protein or RNA.

The term “Cas9” refers to a CRISPR associated endonuclease referred to by this name. Non-limiting exemplary Cas9s are provided herein, e.g., the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

The term “gRNA” or “guide RNA” or “sgRNA” or “single guide RNA” as used herein refers to the guide RNA sequences used to target specific genes for correction employing the CRISPR technique. Techniques of designing gRNAs and donor therapeutic polynucleotides for target specificity are well known in the art. For example, Doench, et al. 2014. Nature biotechnology 32(12):1262-7, Mohr, et al. 2016. FEBS Journal 3232-38, and Graham, et al. 2015. Genome Biol. 16:260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, et al. 2016. J of Biotechnology 233:74-83). As used herein, a biological equivalent of a gRNA includes but is not limited to polynucleotides or targeting molecules that can guide a Cas9 or equivalent thereof to a specific polynucleotide sequence such as a specific region of a cell's genome.

The term “embryo” refers to the early stage of development of a multicellular organism. In general, in organisms that reproduce sexually, embryonic development refers to the portion of the life cycle that begins just after fertilization and continues through the formation of body structures, such as tissues and organs. Each embryo starts development as a zygote, a single cell resulting from the fusion of gametes (i.e., fertilization of a female egg cell by a male sperm cell). In the first stages of embryonic development, a single-celled zygote undergoes many rapid cell divisions, called cleavage, to form a blastula. In some embodiments, the embryo is human. In some embodiments, the embryo is a non-human animal.

The term “mosaicism” is defined as the presence of two or more populations of cells with different genotypes in one individual who has developed from a fertilized egg.

Disclosed herein are agents and methods of controlling gene-editing and regulating CRISPR activity in embryos. The methods disclosed herein utilize anti-CRISPR agents as well as use of these agents in a temporal fashion to control gene editing and regulate CRISPR activity.

The genome bestowed at fertilization determines much of our health as adults. While genetic mutations may be corrected after birth using somatic gene therapy, the efficacy of this approach is dependent on the number of cells that can be edited. In addition, the mutation will still be passed on to the next generation. In contrast, gene editing in the embryo will alter the genome of all cells and can result in a durable therapeutic effect.

An important requirement for clinical application is the ability to predict outcomes. Mosaicism prevents inferring the genotype of the fetus from trophectoderm and is thus incompatible with clinical use. It has previously been shown by the inventor that the activity of off-target sites appears to occur with a delay relative to the on-target site in human embryos. While the majority of on-target sites were modified within the first cell cycle, most off-target genetic changes occurred in later cell cycles and were mosaic (Zuccaro et al. 2020; see also WO 2021/072361, incorporated by reference in its entirety herein). Therefore, the application of CRISPR/Cas9 prior to the first round of DNA replication in the embryo and the introduction of an anti-CRISPR agent after the gene editing, potentially in advance of the replication of the targeted locus or allele, allows potential mosaicism and/or off-target effects to be reduced, eliminated, or prevented, and/or gene editing efficiency to be increased.

The results disclosed herein show that anti-CRISPR protein AcrIIA4 has a potent inhibitory effect on Cas9-mediated gene editing in human embryos when introduced simultaneously at the time of ICSI. Although indels and chromosomal losses were decreased with AcrIIA4, rates of mosaicism in mutant embryos were high, illustrating a delay in the start of Cas9 activity. These data show that Cas9 begins cleaving around 12 hours post-fertilization continues beyond the 16-hour mark, predominantly after the first DNA replication cycle (Balikier et al. 2017).

In an effort to prevent prolonged Cas9 activity, AcrIIA4 was injected into fertilized eggs at the time point in which the majority of on target Cas9-mediated cleavage has occurred. These data demonstrate that a delayed introduction of AcrIIA4 into human embryos injected with Cas9 reduces on-target cleavage by approximately 45% and off-target indels by approximately 74-79%.

Chromosomal segmental losses are also significantly reduced at off-target sites when AcrIIA4 is injected at approximately 16 hours post-fertilization. These data point to differences in the timing of cleavage between on-target and off-target sites.

### Anti-CRISPR Agents

Disclosed herein is the use of anti-CRISPR agents to control gene editing, reduce and/or inhibit gene editing, reduce, eliminate and/or prevent mosaicism and/or off-target effects of gene editing, and/or increase efficiency of gene editing, in embryos, in which gene editing, e.g., CRISPR, systems have been introduced to correct mutations.

Anti-CRISPR agents include but are not limited to anti-CRISPR proteins, either known or newly discovered, and nucleic acids which encode the anti-CRISPR proteins.

To date, scientists have discovered more than 50 distinct families of anti-CRISPR proteins. All known anti-CRISPR proteins are small, typically 50-150 amino acids in size. Some suppress the Cas enzyme's ability to bind to DNA, whereas others prevent the system from cleaving DNA or interfere with the guide RNAs it relies on.

In some embodiments, the anti-CRISPR protein is selected from the group consisting of AcrIIA2 and AcrIIA4.

In some embodiments, the anti-CRISPR protein includes but is not limited to AcrE1, AcrE2, AcreF3 and AcrE4.

In some embodiments, the anti-CRISPR protein includes but is not limited to AcrF1, AcrF2, AcrF3, AcrF4, AcrF5, AcrF6, AcrF7, AcrF8, AcrF9 and AcrF10.

In some embodiments, the anti-CRISPR protein includes but is not limited to AcrIIC1, AcrIIC2 and AcrIIC3.

In some embodiments, the anti-CRISPR protein includes but is not limited to AcrIIA1, AcrIIA2, AcrIIA3 and AcrIIA4.

In some embodiments, the anti-CRISPR protein is a protein listed in Table 1. In some embodiments, the anti-CRISPR protein is a biological equivalent of a protein listed in Table 1. In some embodiment, the anti-CRISPR protein is a variant of a protein listed in Table 1.

Table 1 summarizes the anti-CRISPR proteins known to date.

TABLE 1
Anti-CRISPR proteins

| Name of acr | Type | |
|---|---|---|
| protein | Inhibited | Species of origin |
| AcrIA1 | I-A | Sulfolobus virus Ragged Hills |
| AcrIB1* | I-B | Leptotrichia buccalis DSM 1135 |
| AcrIC1 | I-C | Moraxella bovoculi |
| AcrIF2/C2 | I-C, I-F | Pseudomonas aeruginosa |
| AcrIC3 | I-C | Pseudomonas aeruginosa |
| AcrIC4 | I-C | Pseudomonas aeruginosa |
| AcrIC5 | I-C | Pseudomonas aeruginosa |
| AcrIC6 | I-C, I-E | Pseudomonas aeruginosa |
| AcrIC7 | I-C, I-E | Pseudomonas stutzeri |
| AcrIC8 | I-C, I-E | Pseudomonas aeruginosa |
| AcrIC9 | I-C | Rhodobacter capsulatus |
| AcrIC10 | I-C | Xanthomonas translucens |
| AcrID1 | I-D | Sulfolobus islandicus |
| AcrIE1 | I-E | Pseudomonas aeruginosa |
| AcrIE2 | I-E | Pseudomonas aeruginosa |
| AcrIE3 | T-E | Pseudomonas aeruginosa |
| AcrIE4 | I-E | Pseudomonas aeruginosa |
| AcrIE5 | T-E | Pseudomonas aeruginosa |
| AcrIE6 | I-E | Pseudomonas aeruginosa |
| AcrIE7 | I-E | Pseudomonas aeruginosa |
| AcrIE4-IF7 | I-E/I-F | Pseudomonas aeruginosa |
| AcrIE8 | I-E | Klebsiella michiganensis |
| AcrIE9 | I-E | Pseudomonas sp. |
| AcrIF1 | I-F | Pseudomonas aeruginosa |
| AcrIF2* | I-F | Pseudomonas aeruginosa |
| AcrIF3 | I-F | Pseudomonas aeruginosa |
| AcrIF4 | I-F | Pseudomonas aeruginosa |
| AcrIF5 | I-F | Pseudomonas aeruginosa |
| AcrIF6 | I-F/I-E | Pseudomonas aeruginosa |
| AcrIF7 | I-F | Pseudomonas aeruginosa |
| AcrIF8 | I-F | Pectobacterium carotovorum |
| AcrIF9 | I-F | Vibrio parahaemolyticus |
| AcrIF10 | I-F | Shewanella xiamenensis |
| AcrIF11 | I-F | Pseudomonas aeruginosa |
| AcrIF12 | I-F | Pseudomonas aeruginosa |
| AcrIF13 | I-F | Moraxella catarrhalis |
| AcrIF14 | I-F | Moraxella catarrhalis |
| AcrIE4-IF7 | I-E/I-F | Pseudomonas aeruginosa |
| AcrIF15 | I-F | Klebsiella michiganensis |
| AcrIF16 | I-F | Pectobacterium parmentieri |
| AcrIF17 | I-F | Pectobacterium carotovorum |
| AcrIF18* | I-F/I-E | Serratia marcescens |
| AcrIF19 | I-F | Pectobacterium carotovorum |
| AcrIF20 | I-F | Pectobacterium carotovorum |
| AcrIF21 | I-F | Pectobacterium carotovorum |
| AcrIF22* | I-F/I-E | Pectobacterium parmentieri |
| AcrIF23 | I-F | Pseudomonas aeruginosa |
| AcrIF24 | I-F | Pseudomonas aeruginosa |
| AcrIIA1 | II-A | Listeria monocytogenes |
| AcrIIA2 | II-A | Listeria monocytogenes |
| AcrIIA3 | II-A | Listeria monocytogenes |
| AcrIIA4 | II-A | Listeria monocytogenes |
| AcrIIA5 | II-A | Streptococcus thermophilus |
| AcrIIA6 | II-A | Streptococcus thermophilus |
| AcrIIA7 | II-A | Metagenomic libraries from human |
| | | gut |
| AcrIIA8 | II-A | Metagenomic libraries from human |
| | | gut |
| AcrIIA9 | II-A | Metagenomic libraries from human |
| | | gut |
| AcrIIA10 | II-A | Metagenomic libraries from soil |
| AcrIIA11 | II-A | Clostridium sp. from human gut |
| | | metagenome |
| AcrIIA12 | II-A | Listeria monocytogenes |
| AcrIIA13 | II-A | Staphylococcus schleiferi |
| AcrIIA14 | II-A | Staphylococcus simulans |
| AcrIIA15 | II-A | Staphylococcus delphini |
| AcrIIA16 | II-A | Listeria monocytogenes |
| AcrIIA17 | II-A | Enterococcus faecalis |
| AcrIIA18 | II-A | Streptococcus macedonicus |
| AcrIIA19 | II-A | Staphylococcus simulans |
| AcrIIA20 | II-A | Streptococcus iniae |
| AcrIIA21 | II-A | Streptococcus agalactiae |
| AcrIIA22 | II-A | Clostridium sp. from human gut |
| | | metagenome |
| AcrIIA23 | II-A | Streptococcus pyogenes |
| AcrIIA24 | II-A | Streptococcus phage |
| AcrIIA25 | II-A | Streptococcus phage |
| AcrIIA26 | II-A | Streptococcus sp. from human oral |
| | | metagenome |
| AcrIIA27 | II-A | Streptococcus pyogenes |
| AcrIIA28 | II-A | Streptococcus phage |
| AcrIIA29 | II-A | Streptococcus pyogenes |
| AcrIIA30 | II-A | Streptococcus gordonii |
| AcrIIA31 | II-A | Streptococcus sp. |
| AcrIIA32 | II-A | Streptococcus uberis |
| AcrIIC1 | II-C | Brackiella oedipodis |
| AcrIIC2 | II-C | Neisseria meningitidis |
| AcrIIC3 | II-C | Neisseria meningitidis |
| AcrIIC4 | II-C | Haemophilus parainfluenza |
| AcrIIC5 | II-C | Simonsiella muelleri |
| AcrIIC6 | II-C | Pasteurella multocida |
| AcrID1 | I-D | Sulfolobus islandicus |
| AcrIIIB1 | III-B | Sulfolobus islandicus |
| AcrIII-1 | Any utilising | Sulfolobus islandicus and |
| | cA4 as a | any other species |
| | second | harbouring type III |
| | messenger | CRISPR-Cas |
| AcrVA1 | V-A | Moraxella bovoculi |
| AcrVA2 | V-A | Moraxella bovoculi |
| AcrVA3 | V-A, I-C | Moraxella bovoculi |
| AcrVA4 | V-A | Moraxella bovoculi |
| AcrVA5 | V-A | Moraxella bovoculi |
| AcrVIA1(Lwa)+ | VI-A | Leptotrichia wadei F0279 |
| AcrVIA2+ | VI-A | Leptotrichia wadei F0279 |
| AcrVIA3+ | VI-A | Leptotrichia wadei F0279 |
| AcrVIA4+ | VI-A | Leptotrichia wadei F0279 |
| AcrVIA5+ | VI-A | Leptotrichia wadei F0279 |
| AcrVIA6+ | VI-A | Rhodobacter capsulat R121 |
| AcrVIA7+ | VI-A | Leptotrichia buccalis DSM 1135 |
| AcrVIA1(Lse) | VI-A | Listeria seeligeri LS46 |
| AcrVIB1 | VI-B | Riemerella anatipestifer |
| — | — | — |
| aca1 | I-E, I-F | Pseudomonas aeruginosa |
| aca2 | I-F, II-C | Oceanimonas smirnovii |
| aca3 | II-C | Neisseria meningitidis |
| aca4 | I-F | Pseudomonas aeruginosa |
| aca5 | I-F | Pseudomonas aeruginosa |
| aca6 | I-F | Pseudomonas aeruginosa |
| aca7 | I-F | Pseudomonas aeruginosa |
| aca8 | I-D | Sulfolobus islandicus rod-shaped |
| | | virus 2 |
| aca9 | I-F/I-E | Klebsiella pneumoniae |
| aca10 | I-C/I-E | Pseudomonas citronellolis |
| aca11 | II-A | Streptococcus sp. |
| aca12 | II-A | Streptococcus sp. |
| aca13 | II-A | Streptococcus equinus |

In Class 1 systems, nucleic acid recognition and cleavage are carried out by a complex of gRNA and at least three different Cas proteins. In Class 2 systems these functions are mediated by a single Cas protein bound to gRNA. The Class 2 systems include the CRISPR-Cas9 systems that are widely used for genome editing applications. Anti-CRISPRs have been found that block CRISPR-Cas types of both classes.

Anti-CRISPR proteins inhibit CRISPR-Cas systems at distinct stages. For example, Type I inhibitors, AcrIF1, AcrIF2, AcrIF4, and AcrIF10 prevent gRNA-guided-Cascade from interacting with DNA. AcrIF3 and AcrIE1 disable Cas3 to prevent target cleavage. Type II inhibitors, AcrIIC2 inhibits gRNA loading into Cas9 preventing proper complex assembly, and AcrIIA2, AcrIIA4, AcrIIC3, AcrIIC4, and AcrIIC5 prevent the complex from recognizing target DNA. Following target recognition, Cas9 creates a double stranded DNA break target, leading to its destruction. AcrIIC1 inhibits the nuclease activity of Cas9 to prevent target cleavage. Type V inhibitors, AcrVA1, AcrVA4, and AcrVA5 prevent DNA recognition.

Importantly, AcrIIA2 and AcrIIA4 were able to inhibit the Streptococcus pyogenes (Spy) CRISPR-Cas9 system, which is by far the most widely used system for genome editing applications. This inhibition was demonstrated both in a bacterial assay, and in human tissue culture cells (Shin et al. 2017).

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the polynucleotide is DNA. In some embodiments, the polynucleotide is RNA.

In some embodiments, the anti-CRISPR agent is a small molecule. In some embodiments, the small molecule is BRD0539.

See generally, Davidson et al. 2020; Marino et al. 2020.

Introduction of the anti-CRISPR protein can be accomplished in different ways. In some embodiments, the anti-CRISPR protein can be introduced into the cell or embryo themselves directly. Any method of introduction of polypeptides into cells can be used.

In some embodiments, a polynucleotide encoding the anti-CRISPR protein is introduced into the cell or embryo.

In such cases, the polynucleotide encoding the anti-CRISPR protein can be operably linked to promoter control sequence for expression of the anti-CRISPR protein in the cell or embryo of interest.

The polynucleotide encoding the anti-CRISPR protein can be linear or circular. In some embodiments, the polynucleotide encoding the anti-CRISPR protein can be part of a vector. Suitable vectors include plasmid vectors, phagemids, cosmids, artificial/mini-chromosomes, transposons, and viral vectors. In some embodiments, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

The anti-CRISPR protein or the polynucleotide encoding the anti-CRISPR protein can be introduced into an embryo by a variety of means. These methods include but are not limited to electroporation, liposomal delivery and nanoparticle delivery.

In some embodiments, the embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001).

In other embodiments, the molecules are introduced into the embryo by microinjection. Typically, the embryo is a fertilized one-cell or two-cell stage embryo.

In some embodiments, the cell or embryo is human. In some embodiments, the cell or embryo is a non-human animal.

### Methods of Controlling Gene Editing Using Anti-CRISPR Agents

Disclosed herein methods of controlling gene editing in an embryo with the use of anti-CRISPR agents. In some cases, the timing of the gene editing as well as the timed use of the anti-CRISPR agent is important in controlling the gene editing, e.g., reducing, eliminating and/or preventing mosaicism and/or reducing, eliminating and/or preventing off-target effects of gene editing.

It has previously been shown by the inventors that the majority of on-target sites were modified using CRISPR/Cas9 within the first cell cycle, and most off-target genetic changes occurred in later cell cycles and were mosaic (Zuccaro et al. 2020). The first cell cycle in a human progresses within a one day window from fertilization to mitosis.

One embodiment of the present disclosure is a method of controlling, reducing or preventing gene editing in an embryo undergoing gene editing of a targeted locus or allele, comprising introducing an anti-CRISPR agent into the embryo after the gene editing.

A further embodiment of the present disclosure is a method of reducing, eliminating and/or preventing mosaicism in an embryo undergoing gene editing of a targeted locus or allele comprising introducing an anti-CRISPR agent into the embryo after the gene editing.

Yet a further embodiment of the present disclosure is a method of reducing, eliminating and/or preventing off-target gene editing in an embryo undergoing gene editing of a targeted locus or allele comprising introducing an anti-CRISPR agent into the embryo after the gene editing.

Yet a further embodiment of the present disclosure is a method of increasing gene editing efficiency in an embryo undergoing gene editing of a targeted locus or allele, comprising introducing an anti-CRISPR agent into the embryo after the gene editing.

In some embodiments, the gene editing is performed in an embryo. In some embodiments, the embryo is human. In some embodiments, the embryo is a non-human animal.

In some embodiments, the gene editing in the embryo takes place in the first cell cycle.

In some embodiments, the introduction of the anti-CRISPR agent is after a time sufficient to allow for the gene editing of a targeted locus or allele. In some embodiments, the gene editing takes place in the first cell cycle. In some embodiments, the introduction of the anti-CRISPR agent is during the first cell cycle. In some embodiments, the introduction of the anti-CRISPR agent is in advance of, or before, the replication of a targeted locus or allele.

In some embodiments, the introduction of the anti-CRISPR agent is about 24 hours after the introduction of the gene editing system or agent into the embryo. In some embodiments, the introduction of the anti-CRISPR agent is less than 24 hours after the introduction of the gene editing system or agent into the embryo. In some embodiments, the introduction of the anti-CRISPR agent is about 12 to about 20 hours after the introduction of the gene editing system or agent into the embryo. In some embodiments, the introduction of the anti-CRISPR agent is about 14 to about 18 hours after the introduction of the gene editing system or agent into the embryo. In some embodiments, the introduction of the anti-CRISPR agent is about 16 to about 18 hours after the introduction of the gene editing system or agent into the embryo. In some embodiments, the introduction of the anti-CRISPR agent is about 16 hours after the introduction of the gene editing system or agent into the embryo.

Alternatively, the introduction of the anti-CRISPR agent is less than one hour after gene editing has taken place in the embryo, or about one hour after gene editing has taken place in the embryo, or about one hour to about four hours after gene editing has taken place in the embryo, or about four hours to about eight hours after gene editing has taken place in the embryo, or about eight hours after gene editing has taken place in the embryo.

In some embodiments, the gene editing is performed using an RNA-guided endonuclease and at least one guide RNA.

In some embodiments, the gene editing is performed using CRISPR technology.

In some embodiments, the CRISPR technology is a CRISPR/Cas9 system.

In some embodiments, the RNA-guided endonuclease and at least one guide RNA are introduced into the cell or embryo in the form of a ribonucleoprotein complex comprising the endonuclease complexed to least one gRNA. Preparation of such RNP complexes are known in the art or can be obtained commercially.

In some embodiments, the RNP is introduced into an oocyte at the same time as a sperm cell. This can be accomplished using intracytoplasmic sperm injection (ICSI).

In some embodiments, the anti-CRISPR agent is selected from the group consisting of small molecules, proteins and polypeptides, and nucleic acids.

In some embodiments, the anti-CRISPR agent is a small molecule. In some embodiments, the small molecule is BRD0539.

In some embodiments, the anti-CRISPR agent is an anti-CRISPR protein. In some embodiments, the anti-CRISPR protein is a biological equivalent of an anti-CRISPR protein. In some embodiments, the anti-CRISPR protein is a variant of an anti-CRISPR protein.

In some embodiments, the anti-CRISPR protein is selected from the group consisting of the proteins listed in Table 1. In some embodiments, the anti-CRISPR protein is a biological equivalent of a protein listed in Table 1. In some embodiment, the anti-CRISPR protein is a variant of a protein listed in Table 1.

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the polynucleotide is DNA. In some embodiments, the polynucleotide is RNA.

In some embodiments, the Cas9 endonuclease originates from Streptococcus pyogenes ((Spy) CRISPR-Cas9 system), and the anti-CRISPR protein is selected from the group consisting of AcrIIA2 and AcrIIA4.

In some embodiments, the anti-CRISPR agent is introduced into the embryo using microinjection.

In some embodiments, the anti-CRISPR agent reduces or inhibits the gene editing by at least 50%. In some embodiments, the anti-CRISPR agent reduces or inhibits the gene editing by at least 60%. In some embodiments, the anti-CRISPR agent reduces or inhibits the gene editing by at least 70%. In some embodiments, the anti-CRISPR agent reduces or inhibits the gene editing by at least 75%. In some embodiments, the anti-CRISPR agent reduces or inhibits the gene editing by at least 80%. In some embodiments, the anti-CRISPR agent reduces or inhibits the gene editing by at least 50%. In some embodiments, the anti-CRISPR agent reduces or inhibits the gene editing by at least 90%. In some embodiments, the anti-CRISPR agent reduces or inhibits the gene editing by at least 95%. In some embodiments, the anti-CRISPR agent reduces or inhibits the gene editing by at least 98%. In some embodiments, the anti-CRISPR agent reduces or inhibits the gene editing by at least 99%. In some embodiments, the anti-CRISPR agent reduces or inhibits the gene editing by at least 100%.

Also disclosed herein is a method of performing gene editing in an embryo comprising introducing into the embryo at least one guide RNA or DNA encoding at least one guide RNA, wherein the guide RNA targets a locus or allele, and an RNA-guided endonuclease, or a nucleic acid encoding the RNA-guided endonuclease, and further comprising introducing an anti-CRISPR agent after the introduction the at least one guide RNA, or DNA encoding the at least one guide RNA and the RNA-guided endonuclease, or the nucleic acid encoding the RNA-guided endonuclease, after a time sufficient to allow for gene editing in the targeted locus or allele, wherein the anti-CRISPR agent inhibits or reduces gene editing effects of the at least one guide RNA, or DNA encoding the at least one guide RNA and/or the RNA-guided endonuclease, or the nucleic acid encoding the RNA-guided endonuclease.

In some embodiments, the gene editing is performed in the embryo to correct a mutation in the targeted locus or allele.

In some embodiments, the mutation has a detectable phenotype.

In some embodiments, the endonuclease introduces a double-stranded break in a targeted site on the mutated allele resulting in the correction or modification of the mutation. In some embodiments, the mutation is a frame-shift mutation. In some embodiments, the frame shift mutation is corrected by deleting nucleotides from the mutated allele. In some embodiments, the frame shift mutation is corrected by adding nucleotides to the mutated allele. The current methods and systems to correct frame shift mutations can be used for any mutant homozygous alleles which have a known phenotype. Since the phenotype is known, a father or mother or subject who has the mutated allele would have the corresponding detectable phenotype. Thus, the identification of the allele corresponding to the phenotype is within the skill of the art.

Phenotypes, i.e., disorders or diseases, that can be corrected using the methods and systems of the current disclosure include but are not limited to mutations in the EYS locus, Duchenne's Muscular Dystrophy (DMD) locus, and cardiac myosin-binding protein C3 (MYBPC3) locus.

In some embodiments, the double-stranded break is repaired by a microhomology-mediated end joining repair process.

In some embodiments, wherein the RNA-guided endonuclease is a Cas nuclease. In some embodiments, the Cas nuclease is Cas9.

In some embodiments, the guide RNA is designed to target at least one single nucleotide polymorphism specific for the mutation.

In some embodiments, the gRNA is designed to target the wild-type allele. In some embodiments, the gRNA is designed to target the mutated but not the wild-type allele. In some embodiments, the mutated allele is the paternal allele. In some embodiments, the mutated and wild-type allele differ at the PAM sequence motif. In some embodiments, the gRNA is designed such that placement results in cleavage between two identical regions of polynucleotides in the mutated allele, which defines the sites of micro-homology. In some embodiments, these regions are about 2 bps to about 3 bps. In some embodiments, these regions are about 3 bps to about 5 bps. In some embodiments, these regions are about 5 bps to about 8 bps. Design of gRNA to meet these parameters is known in the art. gRNA can also be obtained commercially.

The gRNA overlaps the mutation to be specific to the mutant allele. The location of the difference should be as close to the PAM site as possible, ideally within 5, or also within 10 nucleotides from the PAM site. A guide RNA can be tested for its specificity to the SNP by in vitro digestion of PCR products containing the different alleles. The specificity test can also be done in cell lines containing heterozygous for the different SNPs and analysis of indel frequency. The gRNA is also designed to cleave between regions of microhomology that can result in predictable removal of a certain number of nucleotides. The number is determined by the nature of the frame shift mutation. For instance, if one nucleotide is missing due to the mutation, a region of microhomology is sought that is 2, or 5, or 8 bp apart, to result in a novel allele with 1, 2, or 3 amino acids fewer than the wild type allele. Despite this change, this restores the entire rest of the protein and will in many cases be functional. Functionality of the novel protein can be tested in a cell culture or animal model.

In some embodiments, a single gRNA is used.

In some embodiments, the guide RNA can be introduced into the embryo as an RNA molecule. The RNA molecule can be transcribed in vitro. Alternatively, the RNA molecule can be chemically synthesized.

In other embodiments, the guide RNA can be introduced into the embryo as a DNA molecule. In such cases, the DNA encoding the guide RNA can be operably linked to promoter control sequence for expression of the guide RNA in the cell or embryo of interest. For example, the RNA coding sequence can be operably linked to a promoter sequence that is recognized by RNA polymerase III (Pol III). Examples of suitable Pol III promoters include, but are not limited to, mammalian U6 or H1 promoters. In some embodiments, the RNA coding sequence is linked to a human U6 promoter. In other exemplary embodiments, the RNA coding sequence is linked to a human H1 promoter.

The DNA molecule encoding the guide RNA can be linear or circular. In some embodiments, the DNA sequence encoding the guide RNA can be part of a vector. Suitable vectors include plasmid vectors, phagemids, cosmids, artificial/mini-chromosomes, transposons, and viral vectors. In some embodiments, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In embodiments in which both the RNA-guided endonuclease and the guide RNA are introduced into the cell as DNA molecules, each can be part of a separate molecule (e.g., one vector containing endonuclease coding sequence and a second vector containing guide RNA coding sequence) or both can be part of the same molecule (e.g., one vector containing coding (and regulatory) sequence for both the endonuclease and the guide RNA).

The RNA-guided endonuclease(s) (or encoding nucleic acid), and the guide RNA(s) (or encoding DNA), can be introduced into an embryo by a variety of means. In some embodiments, the embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001).

In other embodiments, the molecules are introduced into the embryo by microinjection. Typically, the embryo is a fertilized one-cell or two-cell stage embryo.

In some embodiments, the RNA-guided endonuclease and gRNA are introduced into the cell or embryo in the form of a ribonucleoprotein complex comprising the endonuclease complexed to least one gRNA. Preparation of such RNP complexes are known in the art or can be obtained commercially.

In some embodiments, the RNP is introduced into an oocyte at the same time as a sperm cell. This can be accomplished using intracytoplasmic sperm injection (ICSI).

The method further comprises maintaining the embryo under appropriate conditions such that the guide RNA(s) directs the RNA-guided endonuclease(s) to the targeted site(s) in the allele, and the RNA-guided endonuclease(s) introduce at least one double-stranded break in the allele as well as maintaining the embryo under appropriate conditions to administer the anti-CRISPR agent.

An embryo can be cultured in vitro (e.g., in cell culture). Typically, the embryo is cultured at an appropriate temperature and in appropriate media with the necessary O₂/CO₂ ratio to allow the expression of the RNA endonuclease and guide RNA, if necessary. Suitable non-limiting examples of media include M2, M16, KSOM, BMOC, and HTF media. A skilled artisan will appreciate that culture conditions can and will vary.

In some embodiments, the introduction of the anti-CRISPR agent is during the first cell cycle.

In some embodiments, the introduction of the anti-CRISPR agent is in advance of, or before, the replication of the targeted allele.

In some embodiments, the introduction of the anti-CRISPR agent is about 24 hours after the introduction of the at least one guide RNA, or DNA encoding the at least one guide RNA and the RNA-guided endonuclease, or the nucleic acid encoding the RNA-guided endonuclease.

In some embodiments, the introduction of the anti-CRISPR agent is less than 24 hours after the introduction of the at least one guide RNA, or DNA encoding the at least one guide RNA and the RNA-guided endonuclease, or the nucleic acid encoding the RNA-guided endonuclease.

In some embodiments, the introduction of the anti-CRISPR agent is about 12 to about 20 hours after the introduction of the at least one guide RNA, or DNA encoding the at least one guide RNA and the RNA-guided endonuclease, or the nucleic acid encoding the RNA-guided endonuclease.

In some embodiments, the introduction of the anti-CRISPR agent is about 14 to about 18 hours after the introduction of the at least one guide RNA, or DNA encoding the at least one guide RNA and the RNA-guided endonuclease, or the nucleic acid encoding the RNA-guided endonuclease.

In some embodiments, the introduction of the anti-CRISPR agent is about 16 to about 18 hours after the introduction of the at least one guide RNA, or DNA encoding the at least one guide RNA and the RNA-guided endonuclease, or the nucleic acid encoding the RNA-guided endonuclease.

In some embodiments, the introduction of the anti-CRISPR agent is about 16 hours after the introduction of the at least one guide RNA, or DNA encoding the at least one guide RNA and the RNA-guided endonuclease, or the nucleic acid encoding the RNA-guided endonuclease.

Alternatively, the introduction of the anti-CRISPR agent is less than one hour after gene editing has taken place in the embryo, or about one hour after gene editing has taken place in the embryo, or about one hour to about four hours after gene editing has taken place in the embryo, or about four hours to about eight hours after gene editing has taken place in the embryo, or about eight hours after gene editing has taken place in the embryo.

In some embodiments, the anti-CRISPR agent reduces or inhibits the gene-editing by at least 50%. In some embodiments, the anti-CRISPR agent reduces or inhibits the gene-editing by at least 60%. In some embodiments, the anti-CRISPR agent reduces or inhibits the gene-editing by at least 70%. In some embodiments, the anti-CRISPR agent reduces or inhibits the gene-editing by at least 75%. In some embodiments, the anti-CRISPR agent reduces or inhibits the gene-editing by at least 80%. In some embodiments, the anti-CRISPR agent reduces or inhibits the gene-editing by at least 50%. In some embodiments, the anti-CRISPR agent reduces or 85 inhibits the gene-editing by at least 90%. In some embodiments, the anti-CRISPR agent reduces or inhibits the gene-editing by at least 95%. In some embodiments, the anti-CRISPR agent reduces or inhibits the gene-editing by at least 98%. In some embodiments, the anti-CRISPR agent reduces or inhibits the gene-editing by at least 99%. In some embodiments, the anti-CRISPR agent reduces or inhibits the gene-editing by at least 100%.

In some embodiments, the reduction or inhibition of the gene editing reduces, eliminates and/or prevents off-target effects of the gene editing. In some embodiments, the reduction or inhibition of the gene editing reduces, eliminates and/or prevents mosaicism. In some embodiments, the reduction or inhibition of the gene editing increases the gene editing efficiency in the embryo.

In some embodiments, the anti-CRISPR agent is selected from the group consisting of small molecules, proteins and polypeptides, and nucleic acids.

In some embodiments, the anti-CRISPR agent is a small molecule. In some embodiments, the small molecule is BRD0539.

In some embodiments, the anti-CRISPR agent is an anti-CRISPR protein. In some embodiments, the anti-CRISPR protein is a biological equivalent of an anti-CRISPR protein. In some embodiments, the anti-CRISPR protein is a variant of an anti-CRISPR protein.

In some embodiments, the anti-CRISPR protein is selected from the group consisting of the proteins listed in Table 1. In some embodiments, the anti-CRISPR protein is a biological equivalent of a protein listed in Table 1. In some embodiment, the anti-CRISPR protein is a variant of a protein listed in Table 1.

In some embodiments, the anti-CRISPR agent is a polynucleotide encoding an anti-CRISPR protein. In some embodiments, the polynucleotide encodes a biological equivalent of an anti-CRISPR protein. In some embodiments, the polynucleotide encodes a variant of an anti-CRISPR protein.

In some embodiments, the polynucleotide encodes an anti-CRISPR protein in Table 1. In some embodiments, the polynucleotide encodes a biological equivalent of an anti-CRISPR protein listed in Table 1. In some embodiments, the polynucleotide encodes a variant of an anti-CRISPR protein listed in Table 1.

In some embodiments, the polynucleotide is DNA. In some embodiments, the polynucleotide is RNA.

In some embodiments, the Cas9 endonuclease originates from Streptococcus pyogenes ((Spy) CRISPR-Cas9 system), and the anti-CRISPR protein is selected from the group consisting of AcrIIA2 and AcrIIA4.

In some embodiments, the anti-CRISPR agent is introduced into the embryo using microinjection.

The clinical work flow of using the method herein to correct a mutation in an embryo as well as prevent, reduce and eliminate mosaicism and/or off-target effects of the gene editing would be as follows.
1. Genotyping of the somatic cells of both an egg and sperm donor is performed, typically in a fertility clinic or other clinical setting. The cells are sent to a genotyping facility, which may be in a commercial setting and may be a clinical setting.
   The genotyping is performed to determine: 1. the location of the mutated allele; and 2. the specific mutation on the allele.
2. The next step is to obtain a guide RNA based upon the sequence of the mutated allele.
   This gRNA can be designed as described herein or such gRNA can be obtained commercially.
   An RNP is then produced using the gRNA and an RNA-guided endonuclease (e.g., Cas9) using methods known in the art. Alternatively, the RNP can be obtained commercially.
3. The RNP is then introduced into the oocyte using ICSI at the same time as the donor sperm.
4. After about 16 hours, an anti-CRISPR agent, such as an anti-CRISPR protein (e.g., AcrIIA4), is microinjected into the embryo.
5. The embryo is then cultured to the blastocyst stage and a tropectoderm biopsy is performed to determine the karyotype. Embryos with a normal genotype are implanted. The blastocyst may be frozen prior to implantation.

### CRISPR/Cas and Other Endonucleases

Any suitable nuclease may be used in the present methods and systems. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is a group of enzymes that catalyze the hydrolysis of a single nucleotide from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyzes the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

One aspect of the present disclosure provides RNA-guided endonucleases. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences.

One example of an RNA guided sequence-specific nuclease system that can be used with the methods and compositions described herein includes the CRISPR system (Wiedenheft, et al. 2012 Nature 482:331-338; Jinek, et al. 2012 Science 337:816-821; Mali, et al. 2013 Science 339:823-826; Cong, et al. 2013. Science 339:819-823). The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

It is appreciated by those skilled in the art that gRNAs can be generated for target specificity to target a specific gene, optionally a gene associated with a disease, disorder, or condition. Thus, in combination with Cas9, the guide RNAs facilitate the target specificity of the CRISPR/Cas9 system. Further aspects such as promoter choice, may provide additional mechanisms of achieving target specificity, e.g., selecting a promoter for the guide RNA encoding polynucleotide that facilitates expression in a particular organ or tissue. Accordingly, the selection of suitable gRNAs for the particular disease, disorder, or condition is contemplated herein. In one embodiment, the gRNA hybridizes to a gene or allele that comprises a single nucleotide polymorphism (SNP).

Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, CasX, Cas12e, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus therrmophilus, Streptococcus sp., Nocardiopsis dassonvillei, Streptomyces pristinaespiralis, Streptomyces viridochrornogenes, Streptomyces viridochrornogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the polynucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. In some cases, the dCas9 harbors both D10A and H840A mutations of the amino acid sequence of S. pyogenes Cas9. In some embodiments when a dCas9 has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

The present methods and systems may use CRISPR deletion (CRISPRd). CRISPRd capitalizes on the tendency of DNA repair strategies to default towards NHEJ and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In addition to well characterized CRISPR-Cas system, a new CRISPR enzyme, called Cpf1 (Cas protein 1 of PreFran subtype) may be used in the present methods and systems (Zetsche et al. 2015. Cell). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

The sequence-specific endonuclease of the methods and compositions described here can be engineered, chimeric, or isolated from an organism. Endonucleases can be engineered to recognize a specific DNA sequence, by, e.g., mutagenesis. Seligman et al. 2002 Nucleic Acids Research 30:3870-3879. Combinatorial assembly is a method where protein subunits form different enzymes can be associated or fused. Arnould et al. 2006 Journal of Molecular Biology 355:443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Guide RNA(s) used in the methods of the present disclosure can be designed so that they direct binding of the Cas-gRNA complexes to pre-determined cleavage sites in a genome. In one embodiment, the cleavage sites may be chosen so as to release a fragment or sequence that contains a region of a frame shift mutation. In further embodiment, the cleavage sites may be chosen so as to release a fragment or sequence that contains an extra chromosome.

For Cas family enzyme (such as Cas9) to successfully bind to DNA, the target sequence in the genomic DNA can be complementary to the gRNA sequence and may be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

gRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, gRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed (See Prykhozhij et al. 2015 PLoS ONE 10(3):; Zhu et al. 2014 PLoS ONE 9(9); Xiao et al. 2014 Bioinformatics. January 21 (2014)); Heigwer et al. 2014 Nat Methods 11(2):122-123). Methods and tools for guide RNA design are discussed by Zhu 2015 Frontiers in Biology 10(4):289-296, which is incorporated by reference herein. Additionally, there is a publicly available software tool that can be used to facilitate the design of gRNA(s).

### Kits

The present disclosure also provides kits comprising the reagents for practicing any of the methods disclosed herein.

In one embodiment, the kit includes an anti-CRISPR agent and/or delivery systems of such an agent such as vectors comprising these agents and instructions as to timing of the introduction of the anti-CRISPR agent.

In another embodiment, the kit includes RNA-guided endonucleases (e.g., Cas9) or nucleic acid encoding the endonuclease, gRNA(s) or DNA encoding the gRNAs designed to target an allele, and/or delivery systems of such components such as vectors and RNPs comprising these components in addition to an anti-CRISPR agent or agents and/or delivery systems of such an agent or agents such as vectors comprising these agents and instructions as to timing of the introduction of the anti-CRISPR agent.

In some embodiments, the kit includes Cas9 endonuclease and an AcrIIA2 or AcrIIA4 anti-CRISPR protein.

In some embodiments, the kit may include additional reagents such as those for culturing the embryos and testing the embryos for correction of the targeted allele as well as off-target effects.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-6

The Columbia University Institutional Review Board approved all human procedures and experiments.

### Gametes

Cryopreserved human oocytes, semen samples, and zygotes were anonymously donated from individuals who provided informed consent for use in research. Oocytes were cryopreserved between the years 2012 and 2014 using Cryotech vitrification kit. Sperm samples were cryopreserved between the years 2016 and 2018 using Quinn's washing medium from Cooper Surgical and TYB freezing media from Irvine Scientific. All gametes were stored in liquid nitrogen until use.

### Protein Expression and Purification

A bacterial culture harboring the plasmid #101043 with GST-AcrIIA4 fusion protein was obtained from Addgene. The bacteria were streaked on an LB Agar plates with Ampicillin for overnight incubation at 37° C. Individual colonies were then isolated and resuspended in Luria Broth (LB) with Ampicillin and incubated in a shaking incubator at 37° C. for 18 hours. The bacteria were then diluted 1:100 in LB medium with ampicillin and incubated in a shaking incubator at 37° C. until an Optical Density of 0.4-0.6 was reached. The GST-AcrIIA4 fusion protein was then overexpressed with overnight induction of 0.25 mM isopropyl-β-D-thiogalactopyranoside (Research Products International #156000-1.0) at 20° C. in a shaking incubator. The culture was centrifuged and the supernatant was dumped. The bacterial pellet was resuspended in PBS with 1% Triton and the bacterial cells were lysed using sonication. The fusion protein was then purified by centrifugation followed by the addition of Glutathione Sepharose 4B resin (Cytivia #17-0756-01) to the supernatant. The recovered proteins were washed with PBS three times and centrifuged, keeping only the pellet. The pellet was then washed with cleavage buffer (50 mM Tris-HCl, pH 7.0 (at 25° C.), 150 mM NaCl, 1 mM EDTA, 1 mM dithiothreitol) and digested with PreScission Protease (GenScript Z02799-100) to remove the GST tag by chilling the mixture at 5° C. for 4 hours and separated with centrifugation once more. The purified AcrIIA4 protein was stored in cleavage buffer (50 mM Tris-HCl (pH 7.0), 150 mM NaCl, 1 mM EDTA, and 1 mM dithiothreitol (DTT)) and frozen in multiple aliquots at −20° C.

### Protein Purification Confirmation

Isolated AcrIIa4 protein was run on sodium dodecyl sulfate-polyacrylamide gel electrophoresis (SDS-PAGE) with Coomassie blue staining to confirm protein size. The purified protein was also confirmed using Western Blot with an AcrIIA4 primary antibody and anti-mouse secondary antibody (Cell Signaling Technology #7076S). Protein concentration was determined through a Bicinchoninic acid assay.

### RNP Preparation

2 nmol of single guide RNAs were obtained from Integrated DNA Technologies (IDT). Each gRNA was dissolved in 20 ul of nuclease-free water to make a 100 μM sgRNA concentration. Ribonucleoprotein (RNP) preparation was performed as previously described (Turocy et al. 2022). Briefly, 3 μL of injection buffer (5 mM Tris-HCl, 0.1 mM EDTA, pH 7.8), 2 μL of 63M IDT nlsCas9 v3, and 1.5 μL of 100M sgRNA were combined and incubated at room temperature for 5 minutes. After, 96.5 μL of injection buffer was added to the mix. The solution was then centrifuged at 16000 RCF for 2 minutes and then placed on ice prior to intracytoplasmic sperm injection (ICSI).

1.5 nmol of single guide RNA were obtained from Synthego. Each gRNA was dissolved in 15 μL of nuclease-free 1×TE buffer per manufacturer instructions to a concentration of 100 μM. RNP preparation involved combining 3.125 μL of 20 μM Cas9 V3 (New England Biolabs), 0.776 μL of 100 μM sgRNA and incubation at room temperature for 10 minutes. Thereafter, 46 μL of injection buffer was added. The solution was then centrifuged at 16000 RCF for 2 minutes and placed on ice prior to ICSI.

### Oocyte and Zygote Manipulation

Oocyte manipulations were performed in an inverted Olympus IX71 microscope using Narishige micromanipulators. The platform was heated to 37° C. Frozen oocytes were thawed using Cryotech Warming solution set 205. Cryopreserved sperm were thawed to room temperature for 10 min. Quinn's Sperm Washing Medium was added to the thawed sperm and centrifuged at 300×g for 20 min. A second wash was performed and centrifuged again. The sperm pellet was then suspended in 200 μL of wash media and analyzed for viability. Manipulation dishes consisted of a droplet with Cas9 RNP with or without 12.5 μM of AcrIIA4 diluted in injection buffer. Individual motile sperm were immobilized by pressing the sperm tail with the ICSI micropipette, picked up, and expelled in the Cas9 RNP droplet. The sperm was then picked up again for injection into the oocyte cytoplasm. All eggs were cultured in Global total media in an incubator at 37° C. and 5% CO₂. Pronucleus formation to confirm successful fertilization was done on day 1 after ICSI. Experiments with a delayed injection of ACrIIaa4 were performed between 16-17 hour post-ICSI. The tip of an injection needle was nicked, and small amounts of the AcrIIa4 diluted in injection buffer was injected manually into the oocyte cytoplasm using a Narishige micromanipulator.

### Genome Amplification and Genotyping

Zygotes were collected at 12 or 16 hour post-ICSI, and single blastomeres were collected on day 3 using the inverted Olympus IX71 microscope. Narishige micromanipulators and a zona pellucida laser (Hamilton-Thorne) were used to aid in the collection of the blastomeres. All samples were placed in PCR tubes containing 2 or 4 μL of PBS. REPLI-g single kit (Qiagen) which was used for amplification according to the manufacturer's instructions, (with either a half-reaction (2 μL) or a full reaction for (4 μL)). Genotyping was performed with custom primers flanking each cut-site as listed in Table S2.

PCR was performed using AmpliTaq Gold, and all products were run on a 1.5% agarose gel for visual inspection. All samples with a visible band were submitted to Azenta (Genewiz) for Sanger sequencing. Cas9-mediated gene editing was analyzed using NCBI Blast reports and ICE analysis (Synthego).

### Amplicon Next-Generation Sequencing

Twelve DNA samples with indel variants demonstrating fewer than 10% relative abundance on ICE analysis (Synthego) of Sanger sequencing data were amplified using their respective primers. Each sample underwent a precipitation reaction to purify the DNA. Briefly 1/10 volume of 3M sodium acetate was added to PCR products, followed by 3×volume of 100% ethanol. The solution was mixed and incubated at −20° C. for 20 minutes. The sample was centrifuged at 18000G for 20 minutes as room temperature and the supernatant dumped. The pellet was then washed with 800 μL of 70% Ethanol and centrifuged at 15000 g×20 minutes at room temperature. The supernatant was dumped and the pellet was allowed to air dry on a hot plate for 45 minutes. The pellet was reconstituted in 20 μL of nuclease free water. Concentration was checked with nanodrop and normalized to 20 ng/μL. Samples were sent to Azenta for NGS (next-generation sequencing).

### Next-Generation Sequencing Data Analysis

Each of the twelve samples were analyzed for SNP and INDEL detection using a custom analysis workflow by Azenta. Each sample contained >2,000 reads and every unique variant was quantified as a percentage of total read count. Any read containing an indel abundance greater than 10% on Azenta was deemed an indel. NOS data was concordant with the ICE analysis in samples that revealed a read count greater than 5% on ICE analysis. Any result with an allele demonstrating less than a 5% frequency on ICE was excluded from the analysis to avoid any potential false positive or false negative.

### Genome-Wide SNP Array

Genome-wide SNP array was performed as previously described (Zuccaro et al. 2020). Briefly, embryo and blastomere biopsies were amplified at Columbia University using REPLI-g single cell kit according to the manufacturer's instructions or at Genomic Prediction using ePGT amplification (Treff et al. 2019). Copy number and genotyping analysis were performed using gSUITE software (Genomic Prediction). To determine copy number, raw intensities from the Affymetrix Axiom array were processed and subsequently normalized with a panel of wild type male and female samples. Chromosomal breakpoints were mapped by visual analysis by aligning each chromosome copy number plot with its respective chromosome sequence, with the Cas9 cut site highlighted based on its hg38 coordinates. This allowed us to map if the chromosomal breakpoint began precisely at the Cas9 cut site. A segmental error was defined as the gain or loss of a chromosome segment centromeric or telomeric to the Cas9 cut site coordinate.

### Quantification and Statistical Analysis

Statistical analysis was performed using Fisher's exact test. A p-value of less than 0.05 was used to determine significance.

### Example 2—Anti-CRISPR Protein AcrIIA4 Inhibits Cas9 Activity in Human Embryos

To determine if AcrIIA4 could inhibit CRISPR/Cas9 in developing human embryos, Cas9 ribonucleoprotein (RNP) with single-guide RNAs (sgRNA) was injected into donated oocytes at the time of intracytoplasmic sperm injection (ICSI) with or without AcrIIA4. We designed three unique sgRNAs targeting the Duchenne's Muscular Dystrophy (DMD) locus on Chromosome X, (12) the cardiac myosin-binding protein C3 (MYBPC3) locus on Chromosome 11, (13) and the proximal region of the p arm in Chromosome 16 (chr16: 33,918,576 hg38) using Integrated DNA Technologies (IDTDNA). The chromosome 16 gRNA has three secondary sites with identical DNA sequences, yielding six total sites for analysis. We used multiplexing of three unique RNPs to allow us the ability to monitor a larger number of targets per embryo.

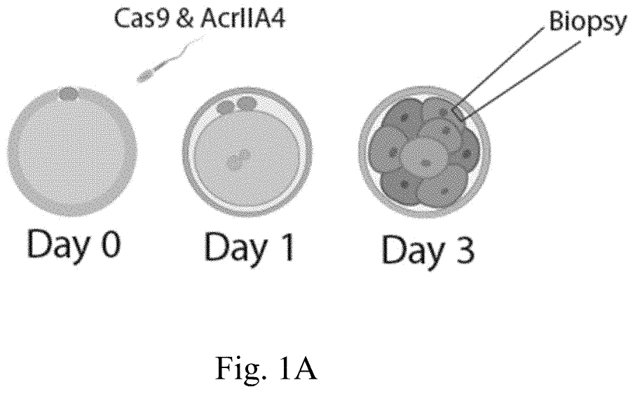

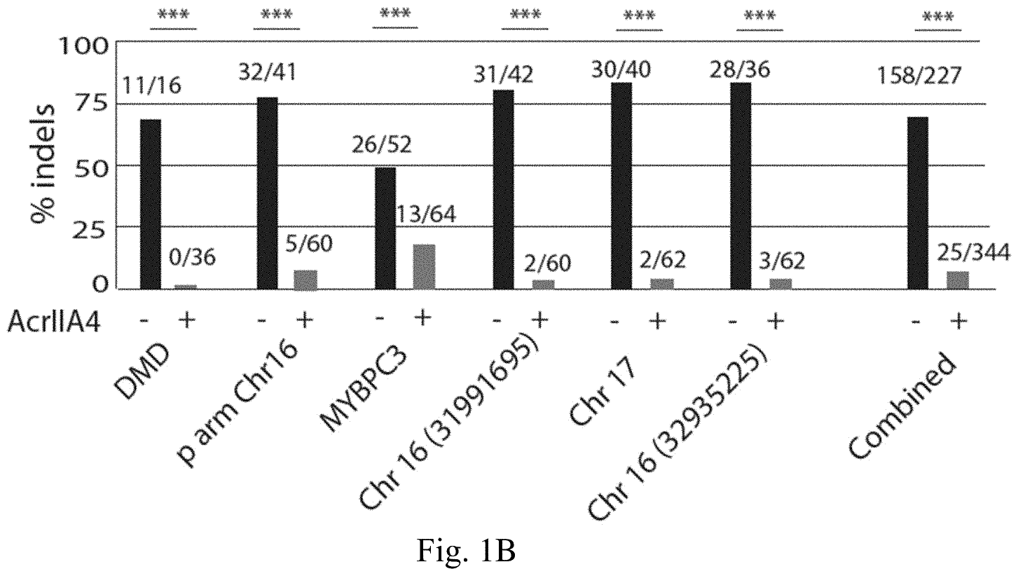

14 donated oocytes were thawed from six donors, along with a single sperm sample from a healthy donor. 10 eggs survived the thaw and were injected with Cas9 RNP using gRNA targeting the DMD, MYBPC3, and the p arm of chromosome 16 loci together with a single sperm. Five oocytes were co-injected with 12.5 μM of AcrIIA4, and five oocytes injected with Cas9 RNP only served as controls (FIG. 1A).

10 out of 10 oocytes fertilized successfully and continued to divide normally. The embryos were cultured and biopsied for genotyping at the cleavage stage on Day 3 after fertilization. A total of 59 blastomeres were generated from 10 embryos (Cas9 RNP only blastomeres (n=27) and Cas9-AcrIIA4 co-injected blastomeres (n=32)). The genomes of individual blastomeres were amplified, used for on target polymerase chain reaction (PCR), and analyzed by Sanger Sequencing. Due to injection of a combination of gRNAs, there were six sites analyzed per blastomere; the three targeted cut sites, as well as three secondary sites which share equivalent target sequences. In addition, the gRNAs were not allele-specific, and thus targeted both homologous chromosomes. Therefore, each target yields two sites for analysis, as it contains two homologous chromosomes 11s, 16s, and 17s, and one or two sites for the DMD locus depending on sex.

All Sanger Sequencing results were analyzed for indels using NCBI Blast reports and ICE analysis (Synthego). ICE analysis quantifies the relative abundance of each allele by assigning all unique variants a percentage of total reads. Alleles are not amplified in a 1:1 ratio due to the stochasticity of amplifying a small number of genetic material (Walsh et al. 1992). To confirm the accuracy of the ICE analysis, twelve samples containing an allele with less than 10% relative abundance were sent for Next Generation Sequencing (NGS) for SNP/INDEL detection, genotyping information, and quantification of unique sequences. NGS data revealed concordance with the ICE analysis in samples that revealed a relative abundance greater than 5% on the initial ICE analysis. Samples with less than 5% allele frequency were classified as background. As such, any result with an allele demonstrating less than a 5% frequency on ICE was excluded from the analysis to avoid any potential false positives.

To assess chromosome copy number and aneuploidies, each sample was analyzed using a high-throughput single nucleotide polymorphism (SNP) array platform validated for Preimplantation Genetic Testing as broken or lost chromosomes are not represented in an on-target PCR and sequencing.

At the DMD locus, 38/59 individual blastomeres amplified and produced a PCR product, yielding 52 Sanger sequencing results (Cas9 only group n=16, Co-injection group n=36). 11/16 alleles (68.7%) revealed an indel at the targeted location in the Cas9 only group vs 0/36 (0%) in the co-injection group (p=<0.0001) (FIG. 1B).

For the chr16:33,918,576 (p arm) locus, 51/59 DNA samples were amplified and produced a PCR product, yielding 101 alleles analyzed via Sanger sequencing (Cas9 only group n=41, Co-injection group n=60). 32/41 alleles revealed an indel at the targeted location the Cas9 only group, vs 5/60 in the co-injection group (78% vs 8.33% respectively p=<0.0001) (FIG. 1B).

58/59 DNA samples amplified and produced a PCR product at the MYBPC3 locus, yielding 116 alleles analyzed via Sanger sequencing (Cas9 only group n=52, Co-injection group n=64). 26/52 alleles revealed an indel at the targeted MYBPC3 location in the Cas9 only group vs. 13/64 in the co-injection group (50% vs 20.3% respectively, p=<0.0001) (FIG. 1B).

The three secondary sites targeted by the chromosome 16 sgRNA yielded 302 alleles that were successfully analyzed via Sanger sequencing. One secondary site on chromosome 16 (chr16:31,991,695 hg38) revealed a 73.8% indel rate (31/42) in the Cas9 only group vs 3.3% (2/60) in the co-injection group (p=<0.00001). The other secondary site on chromosome 16 (chr16: 32,935,225 hg38) revealed a 77.8% indel rate (28/36) vs 4.8% (3/62) in the co-injection group (p=<0.00001). Lastly, a third secondary site on chromosome 17 revealed a 75% indel rate (30/40) vs 3.2% (2/62) in the co-injection group (p=<0.00001) (FIG. 1B).

Cumulatively, 69.6% (158/227) of the alleles analyzed revealed an indel at the targeted location in the Cas9 only group compared to 7.27% (25/334) in the co-injection group (p=<0.0001) (FIG. 1B). These results indicate that AcrIIA4 leads to a 90% reduction in Cas9-mediated indel formation in human embryos when co-injected at the time of ICSI.

### Example 3—Cas9 Cleavage Occurs after the First S-Phase in Human Embryos

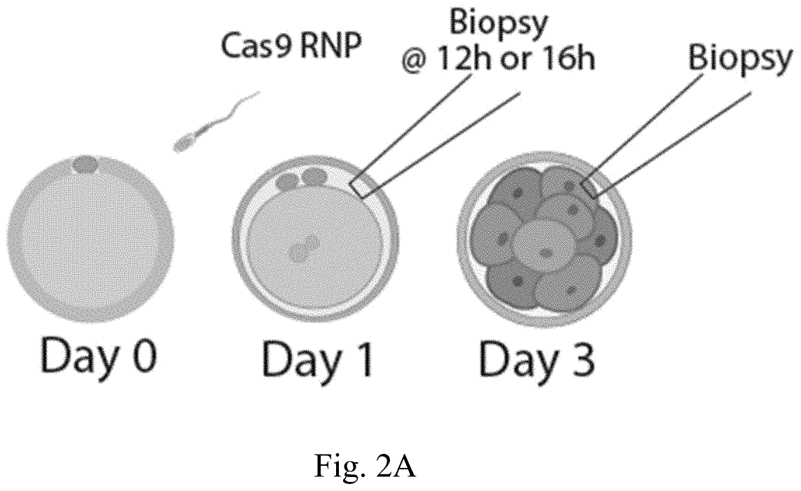

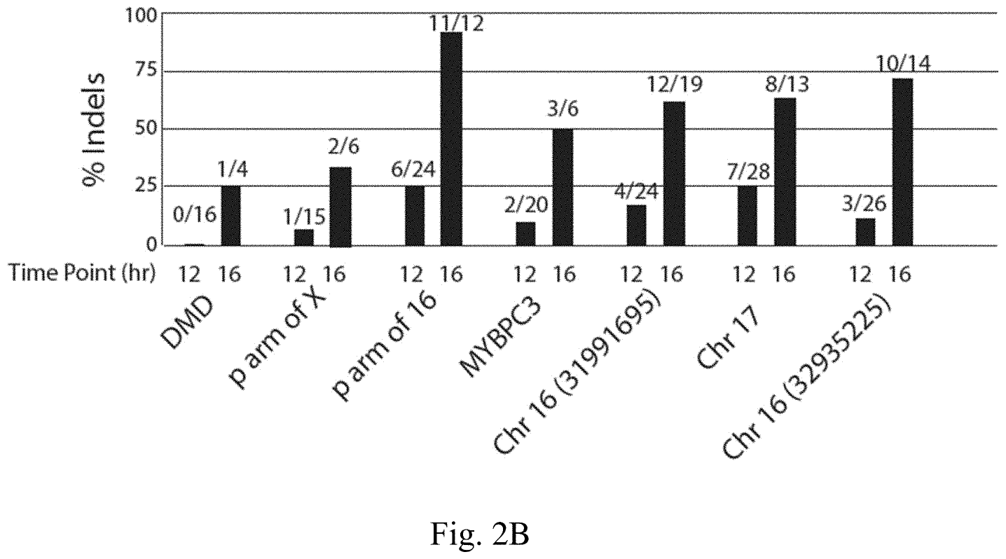

To determine the optimal timing of when to introduce AcrIIA4 into Cas9 exposed zygotes, embryos were harvested at the one cell stage to identify the extent of Cas9 cleavage at different time points. Cas9 RNP with sgRNA was injected into donated oocytes at the time of ICSI and harvested for biopsy and genotyping, between 12-13 and at between 16-17 hours after fertilization (FIG. 2A). 10 donated oocytes were thawed along with one vial of sperm from donors with no known genetic abnormalities. Each egg was injected with the three same sgRNAs: one for the DMD locus on chromosome X; the MYBPC3 locus on chromosome 11; and the p arm chromosome 16 (chr16: 33,918,576 hg38). 9/10 eggs fertilized as indicated by the presence of two pronuclei. Biopsies of either the nucleus, and whenever possible, the polar body were harvested from each zygote at 12 hours (n=5) or 16 hours (n=4) post-fertilization, yielding 17 total samples for analysis (polar bodies n=4, pronuclei n=13). Polar bodies were biopsied to serve as negative controls as mature eggs extrude the first polar body prior to fertilization. As such, polar body sequences should not display any Cas9 modifications. Each biopsy sample was analyzed with Sanger Sequencing for the presence of indels at the three intended Cas9 cut sites, as well as the three secondary cut sites produced by the chromosome 16 sgRNA.

A total of 227 samples produced a PCR product and an adequate Sanger sequence result (12-hour group n=153, 16-hour group n=74). Overall, a significantly higher indel rate was observed at 16 hours compared to 12 hours. At 12 hours, 23/153 (15%) samples revealed an indel at the intended cut-site vs. 47/74 (63.5%) at the 16 hours mark (p=<0.00001). The indel rate at the 16-hour mark was similar to the indel rate observed in which the Cas9-only embryos were harvested at the Day 3 stage (69.6% to 63.5%, respectively, p=0.3) (FIGS. 2A and 2B).

To determine if there is a difference in Cas9 editing efficiency between two commercially available Cas9 enzymes, Cas9 was obtained from New England Biolabs (NEB) along with three new sgRNAs from Synthego. Two of the three sgRNAs targeted the same loci as the IDTDNA gRNAs, including the DMD locus on Chromosome X, the MYBPC3 locus on Chromosome 11. The third guide RNA targeted the Hemoglobin Beta gene (HBB R2) on Chromosome 11. The HBB R2 sgRNA also has a known off-target site on Chromosome 9 at the Glutamate Ionotropic Receptor NMDA Type Subunit 3A (GRIN3A) locus (Cradick et al. 2013). Five donor oocytes were thawed and injected with wild-type sperm and Cas9 RNP using the three new gRNAs. All five oocytes fertilized normally, and individual pronuclei from three zygotes were harvested between 16- and 17 hours to assess indels at that time point. Five pronuclei were successfully isolated from the three zygotes, and each sample was amplified and analyzed for indels using PCR and Sanger Sequencing at all four potential cut sites. The other two zygotes served as controls and were allowed to develop until the cleavage stage before blastomere collection on Day 3. These two embryos yielded 12 blastomeres, which resulted in 63 alleles that were successfully analyzed with Sanger sequencing.

Of the pronuclei collected at the 16 to 17-hour mark, 13.8% (4/29) of alleles analyzed revealed an indel at the Cas9 cut site compared to 68.3% (43/63) in the day 3 Cas9-only blastomeres (FIG. 2C). The Synthego/NEB Cas9 RNP tested revealed a lower indel rate at 16 hours compared to the IDTDNA Cas9 enzyme (13.8% vs 63.5%, respectively) (FIGS. 2C and 2D).

Overall, these data indicate that CRISPR Cas9 editing begins as early as 12 hours and continues beyond the 16 hours post-fertilization, which is after the commencement of the first S-phase in human embryos (Balakier et al. 1993).

### Example 4—Delayed Injection of AcrIIA4 in Zygotes Exposed to CRISPR/Cas9 Reduces Both on and Off Target Editing

To determine if AcrIIA4 can reduce indels and chromosomal changes at off-target sites, three new gRNAs with characterized off-target sites were designed (Zuccaro et al. 2020; Shin et al. 2017; Cradick et al. 2013; Tsai et al. 2015). The gRNAs include targets for the Hemoglobin Beta (HBB) R2 gene on chromosome 11, the Vascular Endothelium Growth Factor (VEGF) 3A gene on chromosome 6, and the Chemokine Receptor 5 (CCRS) gene on chromosome 3. The HBB R2 gRNA has an off-target site on chromosome 9, targeting the GRIN3A gene (Cradick et al 2013). The CCRS gRNA has an off-target site on Chemokine Receptor 2 (CCR2), also on chromosome 3 (Cradick et al. 2013). Lastly, the VEGF3A gRNA has multiple off-target sites, and we focused on the three sites with the highest reported editing rates, two on chromosome 5 and one on chromosome 14 (Tsai et al. 2015). An additional gRNA targeting the p arm of the X chromosome was also utilized, yielding four unique on-target sites and five unique off-target sites for analysis.

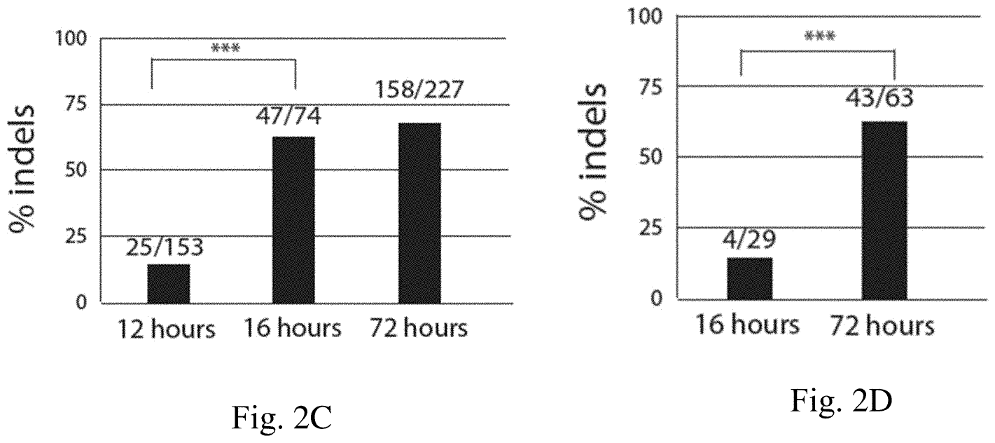

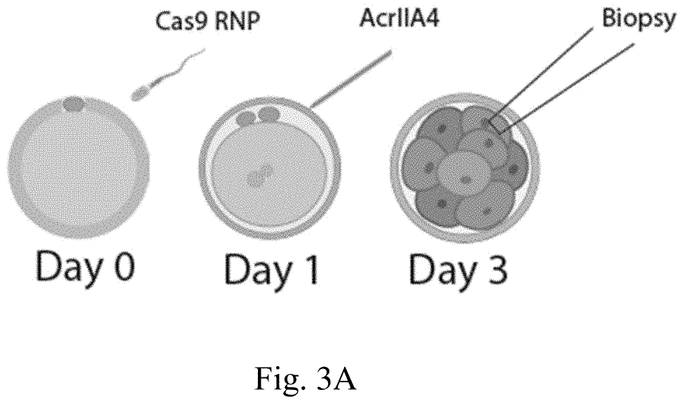

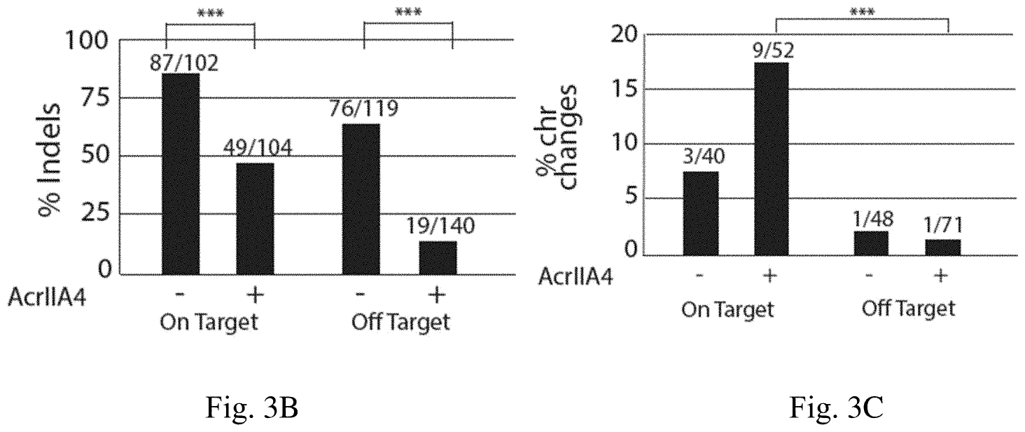

Ten donor oocytes were thawed and injected with wild-type donor sperm along with Cas9 RNP with the four unique sgRNA. 9/10 eggs were fertilized successfully, and four eggs were injected with 12.5 μM of AcrIIA4 at 16-17 hours post-fertilization (FIG. 3A). The 16-hour time point was selected as the Zuccaro 2020 study revealed that on-target Cas9 activity appears to be complete by the 20-hour mark, whereas off-target cleavage had not yet occurred. Also, our earlier analysis also demonstrated high levels of cleavage by 16 hours in the majority of gRNA used from IDTDNA (FIG. 2C). On the third day of embryo cell division, a total of 36 blastomeres were generated from the 10 embryos, and each sample was amplified and analyzed for indels using on target PCR and Sanger Sequencing at all nine target sites. Overall, 290/324 samples yielded a PCR product, providing 465 total alleles that were successfully analyzed via Sanger sequencing (Cas9 only group=221, Cas9+Delayed AcrIIA4 group n=244).

In the Cas9 only group, on-target locations revealed an 85.3% (87/102) indel rate, whereas the off-target locations revealed a 63.9% (76/119) indel rate (p=<0.0001) (FIG. 3B). In the Cas9+Delayed AcrIIA4 group, on-target sites revealed an indel rate of 47.1% (49/104), which demonstrates a 45% reduction of indels compared to the Cas9-only group. The off-target sites revealed an indel rate of 13.6% (19/140), representing a 79% reduction in indel frequency compared to the Cas9-only group (p=0) (FIG. 3B).

Next, we analyzed chromosomal aneuploidy at both the on and off-target sites in both groups. There were significantly fewer segmental changes in the off-target sites compared to the on-target sites in the delayed AcrIIA4 group. Overall, there was a 15.4% (9/52) segmental chromosomal change rate at on-target sites versus a 1.4% (1/71) at the off-target sites in the delayed AcrIIA4 group (p=0.0043) (FIG. 3C). There were no significant differences in chromosomal aneuploidy rates between on and off target sites in the Cas9-only group (FIG. 3C).

Cumulatively, a delayed injection of AcrIIA4 resulted in a 1.7 fold inhibition of total Cas9-induced changes (indels+chromosomal changes) at on-target loci and a 4.86 fold inhibition at off-target loci (p=****) (FIG. 3D).

Nine additional oocytes were thawed to analyze the effects of a delayed AcrIIA4 injection using Synthego gRNAs and NEB Cas9. The sgRNAs targeted the DMD locus on Chromosome X, the MYBPC3 locus on Chromosome 11, and the HBB R2 locus on Chromosome 11. The HBB R2 provided the off-target site on the GRIN3A gene on Chromosome 9. All nine oocytes fertilized and successfully, and four zygotes were injected with 12.5 μM of AcrIIA4 approximately 16 hours post-fertilization. 43 total blastomeres were obtained on Day 3 of embryo development, providing 243 total alleles that were successfully analyzed via Sanger sequencing (Cas9 only group=63, Cas9+Delayed AcrIIA4 group n=180).

In the Cas9 only group, on-target locations revealed an 66.7% (30/45) indel rate, whereas the off-target location revealed a 72.2% (13/18) indel rate (p=NS) (FIG. 3E). In the Cas9+Delayed AcrIIA4 group, on-target sites revealed an indel rate of 37% (47/127), demonstrating a 45% reduction of indels compared to the Cas9-only group (p=<0.00001). The off-target sites revealed an indel rate of 15.1% (8/53) in the delayed AcrIIA4 group, representing a 79% reduction in indel frequency compared to the Cas9-only group (p=0) (FIG. 3E). No significant differences were observed in chromosome copy number changes at on or off target sites between the two groups (FIG. 3F).

Cumulatively, a delayed injection of AcrIIA4 resulted in a 1.8 fold inhibition of total Cas9-induced changes at on-target loci and a 3.9 fold inhibition at off-target loci using the Synthego/NEB Cas9 RNP (p=***) (FIG. 3G).

### Example 5—AcrIIA4 Inhibits Cas9 Activity after the First Cell Cycle

To determine if Cas9 activity persists beyond the first cell cycle, we analyzed if any edited embryo possessed three or more unique genotypes within its blastomeres. Three or more unique genotypes could only occur if there is Cas9 activity after the second S-phase, when there are eight possible alleles to target. Rates of Cas9 activity after the first cell cycle were calculated per chromosomal event and per allele. Only the embryos that contained at least three blastomeres and at least one Cas9-induced change were included in the analysis.

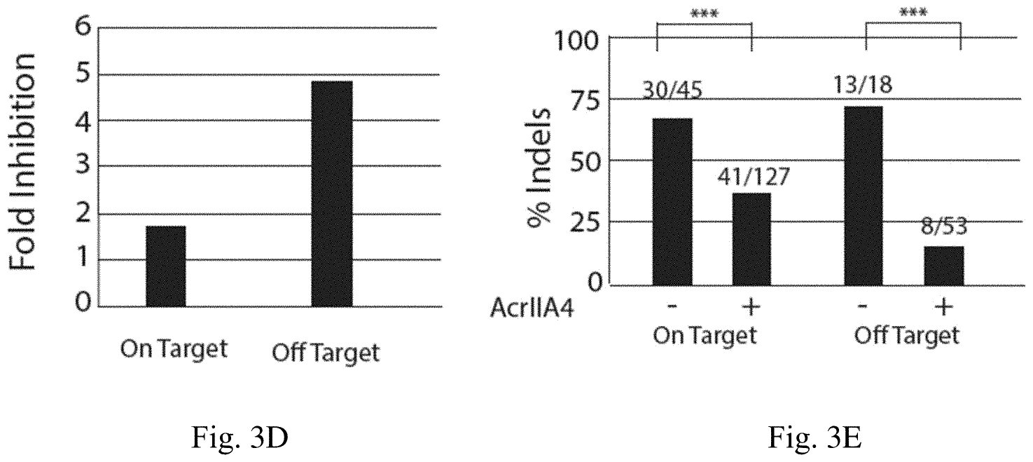

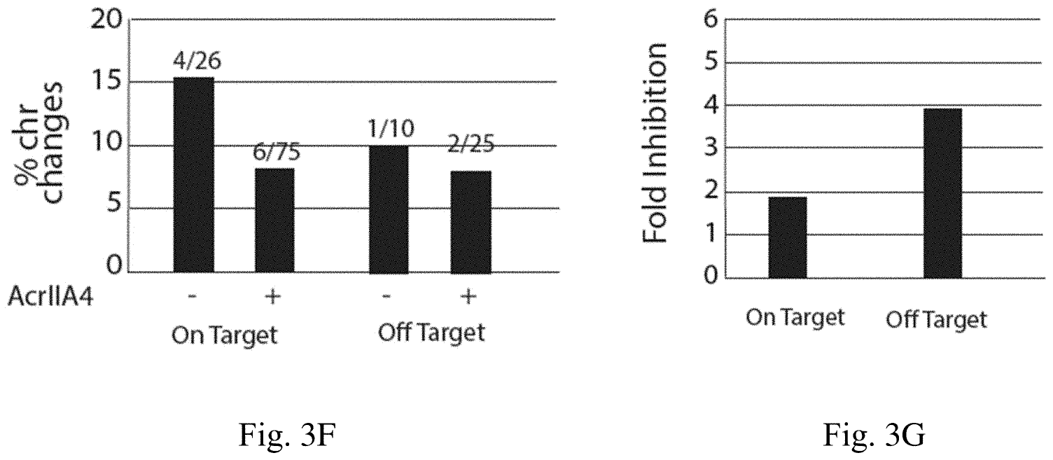

When comparing the Cas9 only embryos versus embryos coinjected with AcrIIA4 Cas9 RNP at six loci, 20/44 (45.5%) alleles revealed evidence of Cas9 activity after the first cell cycle in the Cas9 only group compared to 3/28 (10.7%) in the AcrIIA4 edited embryos (p=0.0016) (FIG. 4A). The delayed introduction of AcrIIA4 approximately 16 hours after fertilization also led to a reduction of Cas9 activity after the first cell division. In the Cas9 only versus the Cas9+delayed AcrIIA4 analysis, 12/29 (41.4%) of alleles revealed Cas9 activity in the first cell cycle compared to 3/22 (13.6%) in the delayed AcrIIA4 group (p=0.06) (FIG. 4B).

### Example 6—AcrIIA4 Reduces Mosaicism Rates

We also analyzed mosaicism rates. Embryos containing blastomeres with two or more unique genotypes were deemed mosaic. High rates of mosaicism indicate that Cas9 activity occurs after the S-phase when the fertilized eggs' DNA has already been replicated. However, if Cas9 activity occurs before the S phase, we would expect all subsequent daughter cells to carry the same uniform edit.

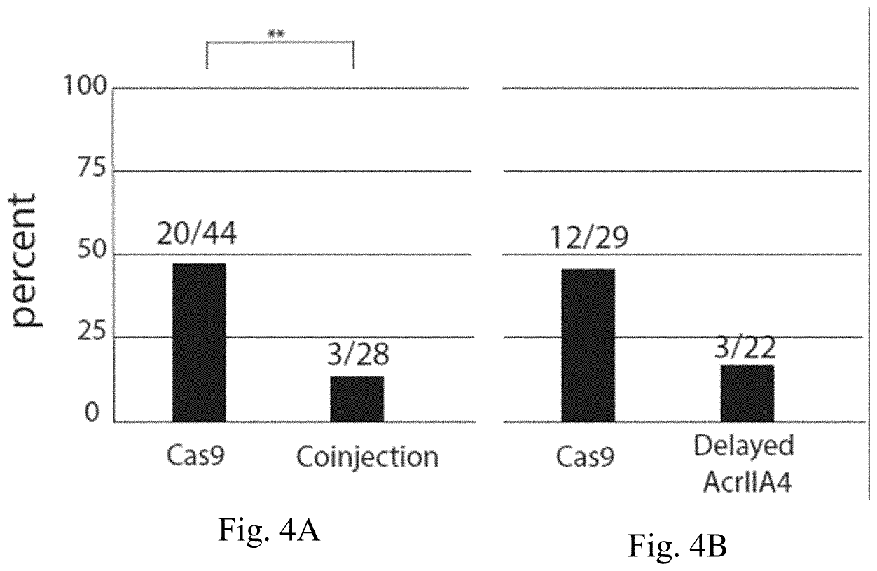

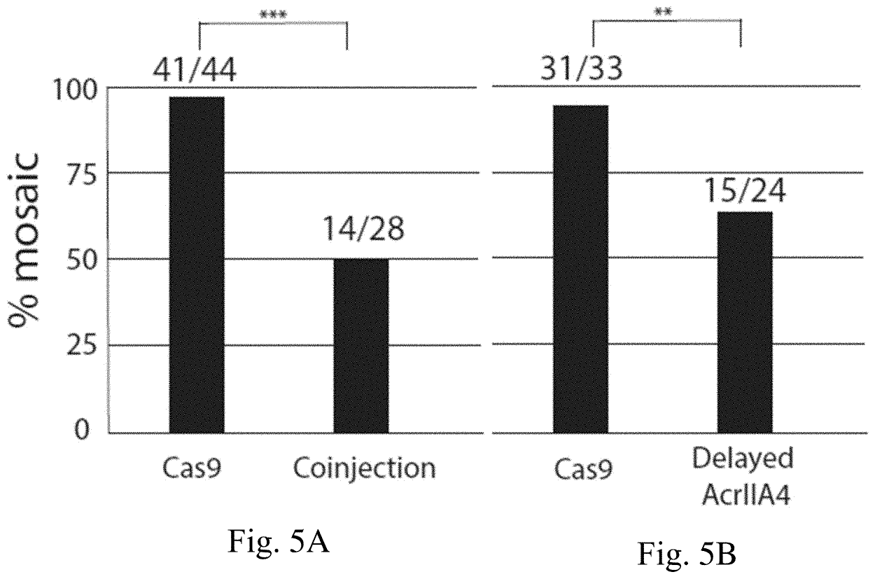

When comparing the Cas9 only embryos versus embryos coinjected with AcrIIA4 Cas9 RNP at six loci, the Cas9 only embryos revealed a 93.2% mosaicism rate in all edited alleles (41/44) whereas the Cas9+AcrIIA4 coinjection group had a 50% (14/28) mosaicism rate (p=0) (FIG. 5A).

When examining the embryos that underwent Cas9 only versus IDTDNA Cas9+delayed AcrIIA4, the Cas9-only group revealed a 93.9% (31/33) mosaicism rate compared to 62.5% (15/24) in the delayed AcrIIA4 group (p=0.0052). (FIG. 5B).

### REFERENCES

Balakier, et al. (1993) Characterization of the first cell cycle in human zygotes: implications for cryopreservation. Fertil Steril. 59(2), 359-65.
Cradick, et al. (2013) CRISPR/Cas9 systems targeting β-globin and CCRS genes have substantial off-target activity. Nucleic Acids Res. 41(20), 9584-92.
Davidson, et al. (2020) Anti-CRISPRs: Protein Inhibitors of CRISPR-Cas Systems. Annual Rev. Biochem. 89, 309-332.
Lee, et al. (2019) Tissue-restricted genome editing in vivo specified by microRNA-repressible anti-CRISPR proteins. Rna. 25(11), 1421-31.
Marino, et al. (2020) Anti-CRISPR protein applications: natural brakes for CRISPR-Cas technologies. Nature Methods 17(5), 471-79.
Mehravar, et al. (2019) Mosaicism in CRISPR/Cas9-mediated genome editing. Dev Biol. 445(2), 156-62.
Pawluk, et al. (2016) Naturally Occurring Off-Switches for CRISPR-Cas9. Cell 167, 1829-1838.e9.
Rodríguez-Rodríguez, et al. (2019) Genome editing: A perspective on the application of CRISPR/Cas9 to study human diseases (Review). Int J Mol Med. 43(4), 1559-74.
Shin, et al. (2017) Disabling Cas9 by an anti-CRISPR DNA mimic. Science Advances 3, e1701620.
Tang, et al. (2017) CRISPR/Cas9-mediated gene editing in human zygotes using Cas9 protein. Mol Genet Genomics 292, 525-533.
Treff, et al. (2019) Validation of concurrent preimplantation genetic testing for polygenic and monogenic disorders, structural rearrangements, and whole and segmental chromosome aneuploidy with a single universal platform. Eur J Med Genet. 62(8), 103647.
Tsai, et al. (2015) GUIDE-seq enables genome-wide profiling of off-target cleavage by CRISPR-Cas nucleases. Nat Biotechnol. 33(2), 187-97.
Tu, et al. (2017) Promoting Cas9 degradation reduces mosaic mutations in non-human primate embryos. Sci Rep. 7,42081.
Turocy, et al. (2021) Heritable human genome editing: Research progress, ethical considerations, and hurdles to clinical practice. Cell 184(6), 1561-74.
Turocy, et al. (2022) DNA Double Strand Breaks cause chromosome loss through sister chromatid tethering in human embryos. bioRxiv. 2022.03.10.483502.
Zuccaro, et al. (2020) Allele-Specific Chromosome Removal after Cas9 Cleavage in Human Embryos. Cell 183, 1650-1664.e15.

1. A method of controlling, reducing or preventing gene editing in an embryo undergoing gene editing of a targeted locus or allele, comprising introducing an anti-CRISPR agent into the embryo after a time sufficient to allow for the gene editing of the targeted locus or allele.

2. The method of claim 1, wherein the method reduces, eliminates or prevents mosaicism of the targeted locus or allele which has been edited and/or reduces, eliminates or prevents off-target effects of the gene editing.

3. The method of claim 1, wherein the gene editing is performed and the introduction of the anti-CRISPR agent is during the first cell cycle.

4. The method of claim 1, wherein the introduction of the anti-CRISPR agent is about 24 hours after the introduction of gene editing system or agent into the embryo.

5. The method of claim 1, wherein the introduction of the anti-CRISPR agent is about 16 to about 18 hours after the introduction of gene editing system or agent into the embryo.

6. The method of claim 1, wherein the introduction of the anti-CRISPR agent is about 16 hours after the introduction of gene editing system or agent into the embryo.

7. The method of claim 1, wherein the anti-CRISPR agent is an anti-CRISPR protein or a polynucleotide encoding an anti-CRISPR protein.

8. The method of claim 7, wherein the anti-CRISPR protein is selected from the group consisting of the proteins listed in Table 1.

9. The method of claim 1, wherein the gene editing is performed using CRISPR technology.

10. The method of claim 1, wherein the gene editing is performed using a CRISPR/Cas9 system and the anti-CRISPR agent is a protein selected from the group consisting of AcrIIA2 and AcrIIA4.

11. A method of performing gene editing in an embryo comprising introducing into the embryo at least one guide RNA or DNA encoding at least one guide RNA, wherein the guide RNA targets a locus or allele, and an RNA-guided endonuclease, or a nucleic acid encoding an RNA-guided endonuclease, and further comprising introducing an anti-CRISPR agent after the introduction the at least one guide RNA, or DNA encoding the at least one guide RNA and the RNA-guided endonuclease, or the nucleic acid encoding the RNA-guided endonuclease, after a time sufficient to allow for gene editing in the targeted locus or allele, wherein the anti-CRISPR agent inhibits or reduces gene editing effects of the at least one guide RNA, or DNA encoding the at least one guide RNA and the RNA-guided endonuclease, or the nucleic acid encoding an RNA-guided endonuclease.

12. The method of claim 11, wherein the method reduces, eliminates or prevents mosaicism of the mutated allele which has been edited and/or reduces, eliminates or prevents off-target effects of the gene editing.

13. The method of claim 11, wherein the at least one guide RNA and the RNA-guided endonuclease are introduced in a ribonucleoprotein complex by intracytoplasmic sperm injection with sperm into an oocyte.

14. The method of claim 11, wherein the introduction of the anti-CRISPR agent is about 24 hours after the introduction of the at least one guide RNA, or DNA encoding the at least one guide RNA and the RNA-guided endonuclease, or the nucleic acid encoding the RNA-guided endonuclease.

15. The method of claim 11, wherein the introduction of the anti-CRISPR agent is about 16 to about 18 hours after the introduction of the at least one guide RNA, or DNA encoding the at least one guide RNA and the RNA-guided endonuclease, or the nucleic acid encoding the RNA-guided endonuclease.

16. The method of claim 11, wherein the introduction of the anti-CRISPR agent is about 16 hours after the introduction of the at least one guide RNA, or DNA encoding the at least one guide RNA and the RNA-guided endonuclease, or the nucleic acid encoding the RNA-guided endonuclease.

17. The method of claim 11, wherein the anti-CRISPR agent is an anti-CRISPR protein or a polynucleotide encoding an anti-CRISPR protein.

18. The method of claim 17, wherein the anti-CRISPR protein is selected from the group consisting of the proteins listed in Table 1.

19. The method of claim 11, wherein the gene editing is performed using a CRISPR/Cas9 system and the anti-CRISPR agent is a protein selected from the group consisting of AcrIIA2 and AcrIIA4.

20. The method of claim 11, wherein the anti-CRISPR agent is introduced into the embryo using microinjection.
